(19) United States
(12) Patent Application Publication
(10) Pub. No.: US 2003/0215405 A1
(43) Pub. Date: Nov. 20, 2003

(54) Cosmetic or dermatological light protection agent preparations

(76) Inventors: Robert Parker, Mannheim (DE); Thomas Heidenfelder, Dannstadt (DE); Gerhard Wagenblast, Wachenheim (DE)

Correspondence Address: Herbert B. Keil, KEIL & WEINKAUF, 1350 Connecticut Ave., N.W., Washington, DC, 20036, US

(21) Appl. No.: 10/289,334

(22) Filed: Nov. 7, 2002

(30) Foreign Application Priority Data
Nov. 12, 2001 (DE) 10155542.3

(51) Int. Cl. A61K 7/42

(52) U.S. Cl. 424/59

(57) ABSTRACT

The invention describes cosmetic or dermatological preparations for skin and hair which comprise at least one cholesteric liquid-crystalline component which reflects in the infrared wavelength range from 750 nm to 2500 nm, at least one component which protects against radiation in the ultraviolet ray range from 280 nm to 450 nm, and in cosmetically acceptable carrier, and to the use thereof for reducing the warming of skin and hair and for protecting against solar rays.

The invention relates to cosmetic or dermatological light protection agent preparations which comprise at least one cholesteric liquid-crystalline component which reflects in the infrared wavelength range from 750 nm to 2500 nm, at least one filter substance which protects against radiation in the ultraviolet ray range from 280 nm to 449 nm and at least one cosmetically acceptable carrier, and to the use thereof.

The light protection agents used in cosmetic and dermatological preparations have the task of preventing, or at least diminishing the extent of, the harmful effects of sunlight on the human skin. In addition, these light protection agents, however, also serve to protect other ingredients from decomposition or degradation by UV radiation. In hair cosmetic formulations, the aim is to prevent damage to the keratin fibers by UV rays.

The sunlight which reaches the earth's surface contains UV-B radiation (280 to 320 nm) and UV-A radiation (above 320 nm), which directly border the visible light region. The effect on human skin is evident, particularly in the case of UV-B radiation, from sunburn. Accordingly, the industry offers a relatively large number of substances which absorb the UV-B radiation and thus prevent sunburn.

The light protection agents often comprise two or more compounds which absorb in the UV range in order to achieve as far-reaching a protection in the UV-A and UV-B range as possible. In addition, light protection agents for cosmetic purposes must also meet a large number of other requirements, for example good solubility in cosmetic oils, high stability of the emulsions prepared therewith, toxicological acceptability, and low intrinsic odor and low intrinsic color.

DE-A-197 45 647 describes thermal insulation coatings for the automobile and construction sector which comprise at least one cholesteric IR-reflecting layer.

DE-A-198 48 130 and DE-A-198 24 972 disclose the use of cholesteric pigments as UV filters in cosmetic and pharmaceutical preparations.

DE-A-196 297 61 describes cosmetic or pharmaceutical preparations which comprise pigments whose color depends on the viewing angle. The pigments comprise at least one oriented crosslinked substance with liquid-crystalline structure and with chiral phase. The pigments used in the cosmetic and pharmaceutical formulations have the disadvantage that the compounds are colored, meaning that it is only possible to use them in cosmetic and pharmaceutical preparations with which a UV protection is achieved to a limited degree.

WO-A-99/11733 describes quite generally a suitability of colored cholesteric pigments for applications in the cosmetic sector. However, use in sunscreens for protection against IR and UV radiation is not described.

The light protection agents customary hitherto do not prevent the skin and hair from perceptively warming up under the influence of solar radiation. This has recently increasingly been regarded as undesirable; a person no longer feels fresh. In addition, it has been found that the thermal radiation can cause damage to the skin and/or the hair; for example, light urticaria are co-triggered by radiation in the infrared wavelength range. There is a therefore a current need for light protection agents which protect the skin and the hair against the action of heat.

It is an object of the present invention to provide cosmetic or dermatological light protection agent preparations which can be prepared easily and which offer effective protection against UV and thermal radiation. The light protection agents should preferably absorb with high absorbance, be photostable and have no, or only a slight, intrinsic color in the visible region, i.e. a sharp band structure.

We have found that this object is achieved by a cosmetic or dermatological preparation which comprises

A) at least one cholesteric liquid-crystalline component which reflects in the infrared wavelength range from 750 nm to 2500 nm,

B) at least one component which protects against radiation in the ultraviolet ray range from 280 nm to 449 nm and

C) a cosmetically or dermatologically acceptable carrier.

The preparation according to the invention comprises, as constituent A), a cholesteric liquid-crystalline component. A prerequisite for the existence of cholesteric phases is the chirality. The chiral molecular moiety can either be pregiven in the liquid-crystalline molecule itself, or else be added as dopant to a nematic phase, as a result of which a chirally nematic phase is induced. In principle, virtually all known cholesteric monomers or monomer mixtures or polymers or polymer mixtures can be adjusted in the pitch of their helical superstructure by variation of the chiral component such that their reflection maximum is in a certain region of the electromagnetic spectrum.

Component A) is preferably chosen from

a) at least one cholesteric polymerizable monomer;

b) at least one achiral, nematic, polymerizable monomer and a chiral compound;

c) at least one cholesteric crosslinkable polymer;

d) at least one cholesteric polymer in a polymerizable diluent or a mixture of polymerizable diluents;

e) at least one cholesteric polymer whose cholesteric phase can be frozen by rapid cooling to below the glass transition temperature; or

f) at least one achiral, liquid-crystalline crosslinkable polymer and a chiral compound;

in each case in a hardened state.

For the purposes of the present invention, crosslinking is to be understood as meaning the covalent linking of polymeric compounds, and polymerization is to be understood as meaning the covalent linking of monomeric compounds to give polymers. Hardening is to be understood as meaning crosslinking, polymerization or the freezing of a cholesteric phase.

Hardening fixes the uniform orientation of the cholesteric molecules in the cholesteric layer.

Suitable cholesteric components A) are described in DE-A-197 45 647, to the entire contents of which reference is hereby made.

Of the abovementioned embodiments a) to f), preference is given 45 to variants a) and b).

Variant a)

In particular, the monomers a) comprise at least one chiral, liquid-crystalline, polymerizable monomer of the formula I

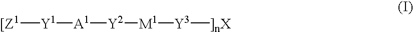

in which the variables have the following meanings:

Z¹ is a polymerizable group or a radical which carries a polymerizable group,

Y¹,Y²,Y³ independently of one another are chemical bonds, oxygen, sulfur,

—CO—O—, —O—CO—, —O—CO—O—,

—CO—S—, —S—CO—,

—CO—N(R)—, —N(R)—CO—, CH₂O, OCH₂,

—N(R)—CO—O— or —O—CO—N(R)—,

A¹ is a spacer,

M¹ is a mesogenic group,

X is an n-valent chiral radical,

R is hydrogen or C₁-C₄-alkyl,

n is 1 to 6

where the radicals Z¹, Y¹, Y², Y³, A¹ and M¹ may be identical or different if n is greater than 1.

Preferred radicals Z¹ are:

CH₂═CH—, CH≡C—, 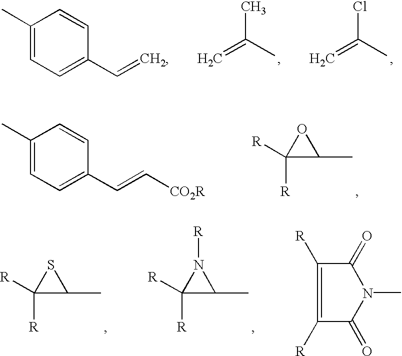
—N═C═O, —N═C═S, —O—C≡N,

—COOH, —OH or —NH₂,

where the radicals R may be identical or different and are hydrogen or C₁-C₄-alkyl, such as methyl, ethyl, n-propyl, isopropyl, n-butyl, isobutyl, sec-butyl or tert-butyl. Of the reactive polymerizable groups, the cyanates can spontaneously trimerize to give cyanurates and are therefore preferred. The other said groups require further compounds with complementary reactive groups for the polymerization. Thus, for example, isocyanates can polymerize with alcohols to give urethanes and with amines to give urea derivatives. Analogous statements apply for thiiranes and aziridines. Carboxyl groups can be condensed to give polyesters and polyamides. The maleimido group is particularly suitable for free radical copolymerization with olefinic compounds such as styrene. The complementary reactive groups may here either be present in a second compound, which is mixed with the first, or they may be incorporated into the polymeric network via auxiliary compounds which contain 2 or more of these complementary groups.

Particularly preferred groups Z¹-Y¹ are acrylate and methacrylate.

Representative spacers A¹ are, for example:

—(CH₂)ₚ—, —(CH₂CH₂O)ₘCH₂CH₂—, —CH₂CH₂SCH₂CH₂—, —CH₂CH₂NHCH₂CH₂—,

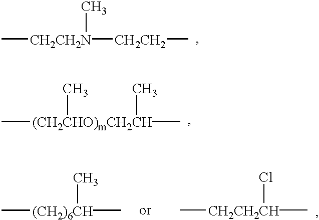

where

m is 1 to 3 and

p is 1 to 12.

The mesogenic group M¹ preferably has the structure

(T-Y⁸ )ₛ-T

where Y⁸ is a bridging member according to one of the definitions of Y¹, s is a number from 1 to 3 and T are identical or different divalent isocycloaliphatic, heterocycloaliphatic, isoaromatic or heteroaromatic radicals. If s is a number >1, the bridging members Y⁸ may be identical or different.

The radicals T may also be ring systems substituted by C₁-C₄-alkyl, fluorine, chlorine, bromine, cyano, hydroxyl or nitro.

Particular preference is given to the following mesogenic groups M¹:

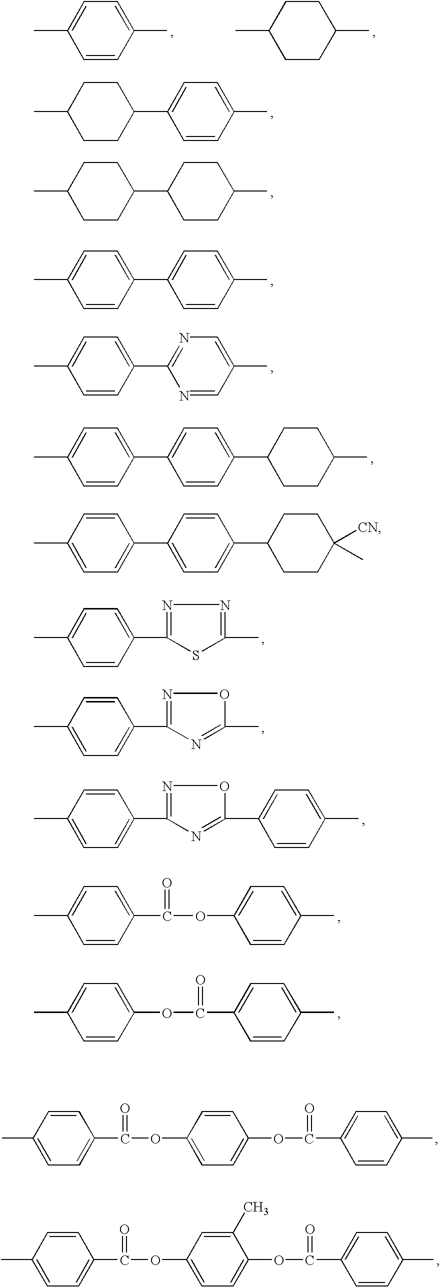

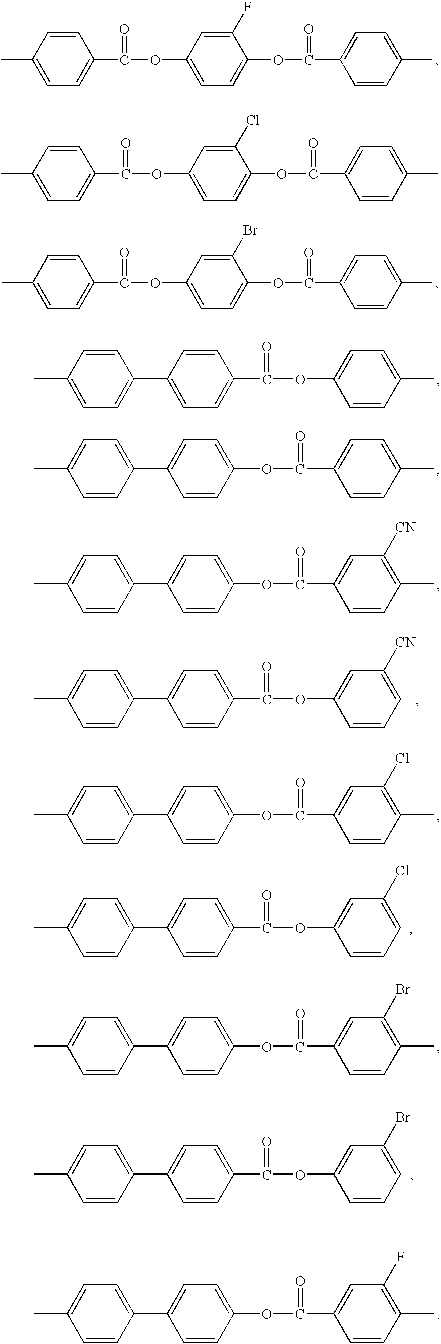

Of the chiral radicals X of the compounds of the formula I, particular preference is given, inter alia, for reasons of availability, to those derived from sugars, binaphthyl or biphenyl derivatives, and optically active glycols, dialcohols or amino acids. Preferred sugars are, in particular, pentoses and hexoses and derivatives derived therefrom.

Examples of radicals X are the following structures, where the terminal lines are in each case the free valences.

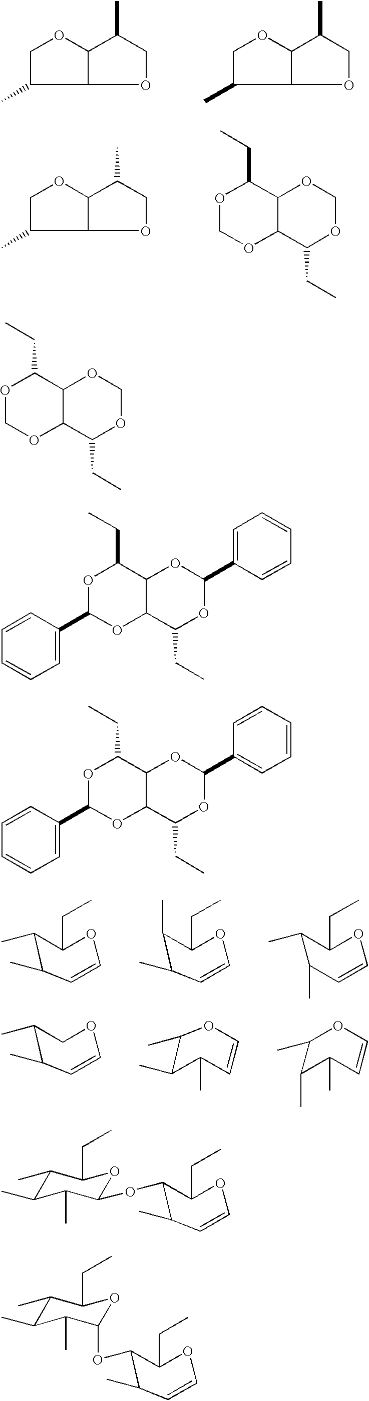

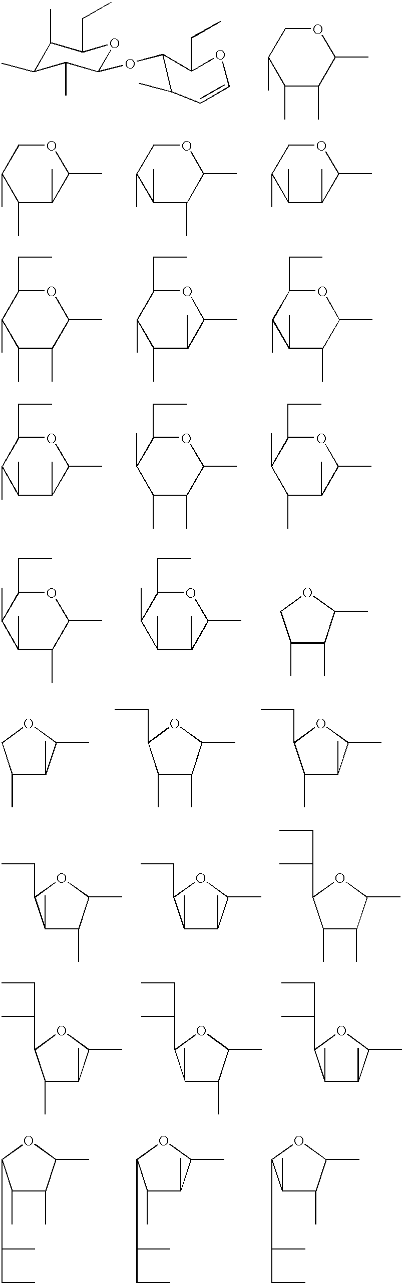

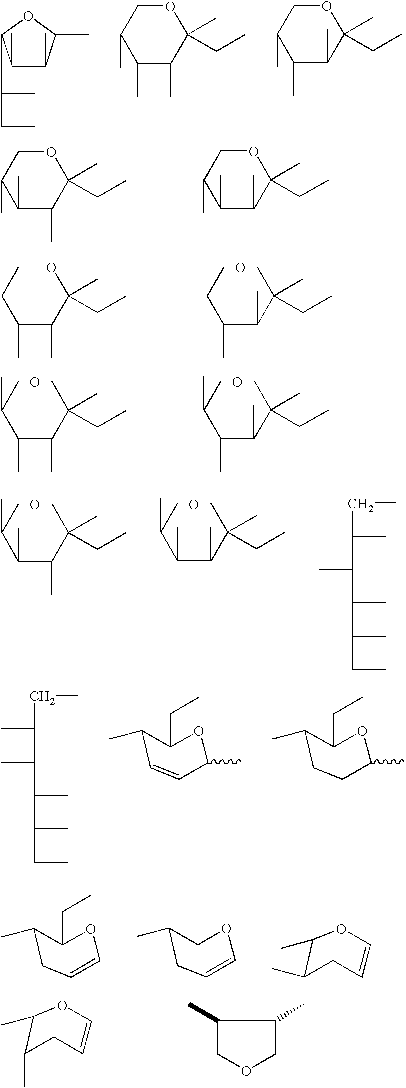

Particular preference is given to

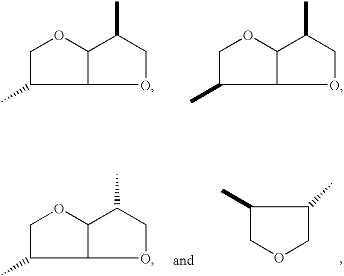

In addition, chiral groups which have the following structures are also suitable:

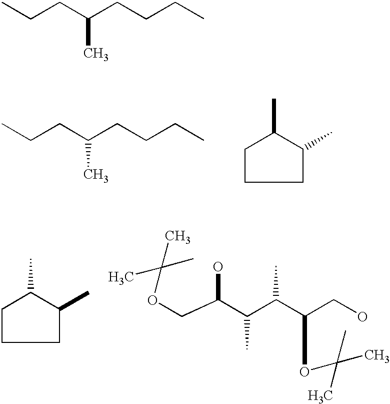

Further examples are listed in German application P 43 42 280.2.

n is preferably 2.

Variant b)

As preferred monomers of group b), the polymerizable mixture comprises at least one achiral nematic, polymerizable monomer of the formula II

Z²-Y⁴-A²-Y⁵-M²-Y⁶-A³-(Y⁷-Z³)_{d} (II)

in which the variables have the following meanings:

Z², Z³ are identical or different polymerizable groups or radicals which contain a polymerizable group

d is 0 or 1

Y⁴, Y⁵, Y⁶, Y⁷ independently of one another are chemical bonds, oxygen, sulfur,

—CO—O—, —O—CO—, —O—CO—O—,

—CO—S—, —S—CO—,

—CO—N(R)—, —N(R)—CO—, CH₂O, OCH₂,

—N(R)—CO—O— or —O—CO—N(R)—

A², A³ are identical or different spacers and

M² is a mesogenic group.

In this connection, for the polymerizable groups, the bridging members Y⁴ to Y⁷, the spacers and the mesogenic group, the same preferred meanings apply as for the corresponding variables of formula I.

In addition, the mixture of group b) comprises a chiral compound. The chiral compound brings about the twisting of the achiral liquid-crystalline phase to give a cholesteric phase. In this connection, the degree of twisting depends on the ability of the chiral dopant to twist and on its concentration. Thus, the pitch of the helix and in turn also the reflection wavelength thus depends on the concentration of the chiral dopant. For this reason, no generally valid concentration range can be given for the dopant. The dopant is added in an amount at which the desired reflection arises.

Preferred chiral compounds are those of the formula Ia

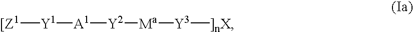

in which Z¹, Y¹, Y², Y³, A¹, X and n have the abovementioned meanings and M^{a} is a divalent radical which contains at least one hetero- or isocyclic ring system.

The molecular moiety M^{a} here is similar to the mesogenic groups described, since particularly good compatibility with the liquid-crystalline compound is achieved in this way. However, M^{a} need not be mesogenic since compound Ia should bring about a corresponding twisting of the liquid-crystalline phase merely through its chiral structure. Preferred ring systems which are present in M^{a} are the abovementioned structures T, and preferred structures M^{a} are those of the abovementioned formula (T-Y⁸)ₛ-T. Further monomers and chiral compounds of group b) are described in WO 97/00600, to the entire contents of which reference is hereby made.

The preparation according to the invention particularly preferably comprises chiral compounds (2A) to (2E) in all of its diastereomeric forms:

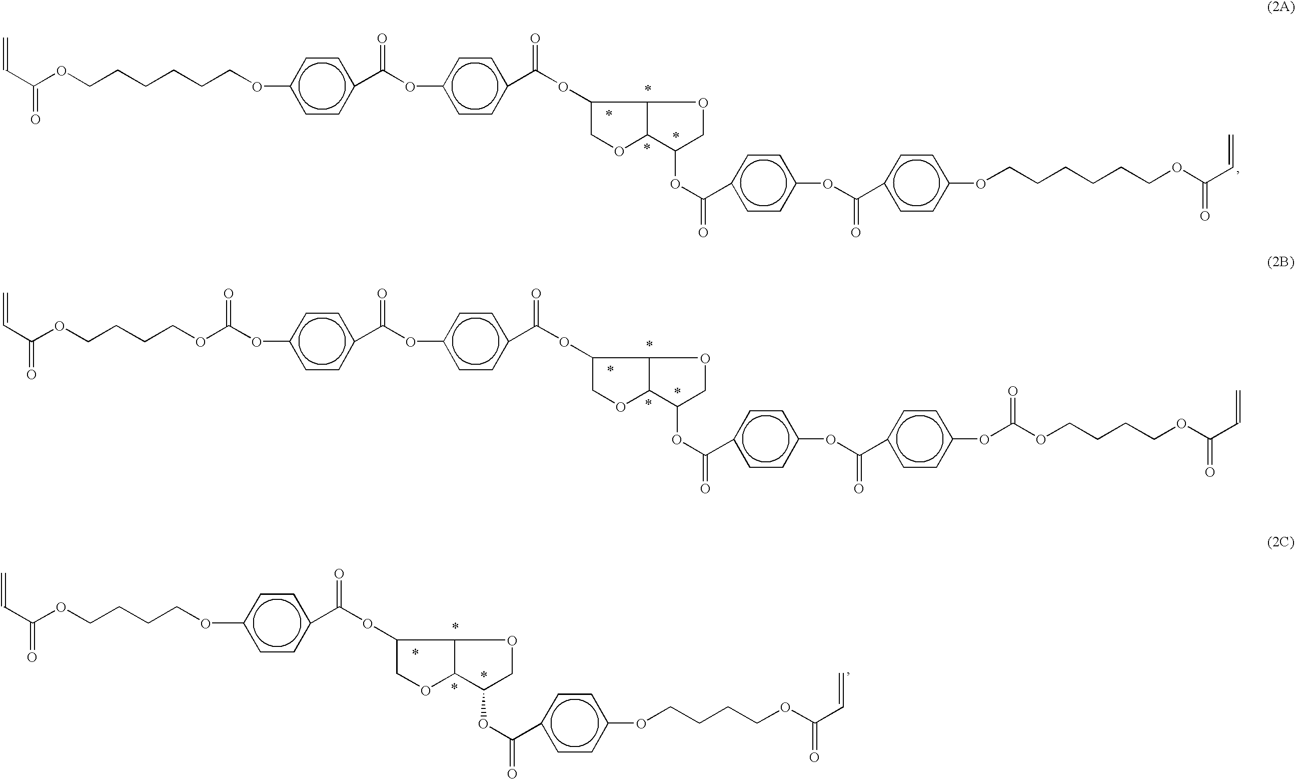

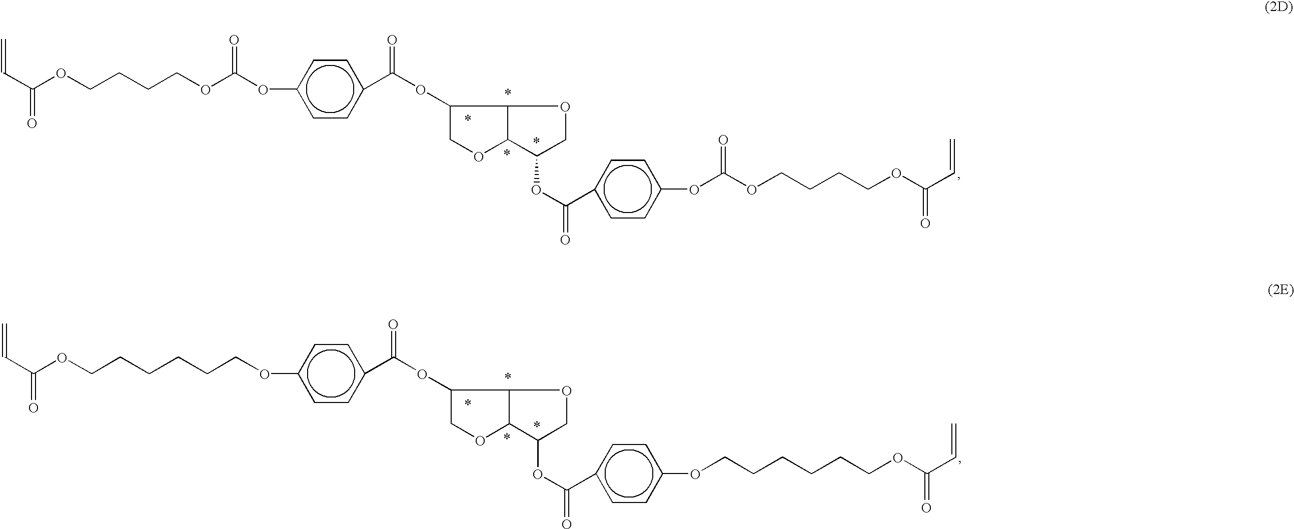

in which * is an asymmetrical carbon atom

and nematic monomers of the formula 1:

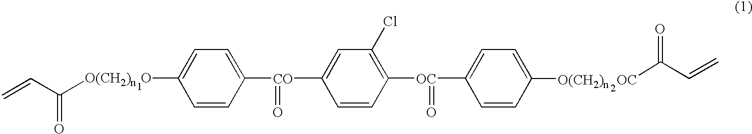

of the formula 3:

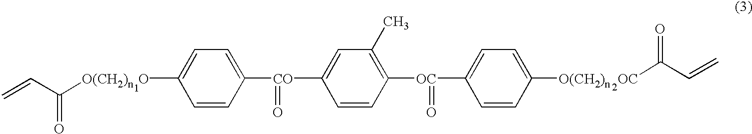

or of the formula 4:

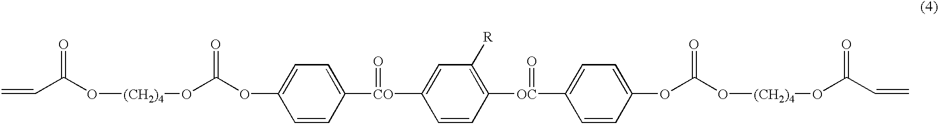

where, in the formulae 1 and 3, n₁ and n₂, independently of one another, are 2, 4 or 6 and the monomers of the formula 1 or 3 are preferably used as mixtures of compounds where n₁/n₂=2/4, 2/6, 4/2, 6/2, 4/4, 4/6, 6/4 or 6/6, and R in formula 4 is H, Cl or CH₃.

Preferably, component A) is used in the form of pigments. To prepare the pigments, the monomers or polymerizable or crosslinkable groups present in the cholesteric component are converted, by free radical or ionic polymerization processes, which can be initiated by a thermal or photochemical reaction, into highly crosslinked polymers with a frozen liquid-crystalline order structure.

The preparation of such pigments is known and is described, inter alia, in detail in WO 99/11733 and in DE-A-197 38 369.

In a preferred embodiment, three-dimensionally crosslinkable polymerizable monomers are applied to a substrate, crosslinked on this substrate and, after crosslinking, detached from the substrate. Preference is given to using the cholesteric material in the composition according to the invention in the form of a finely divided powder. If the polymers are not produced as a finely divided powder in the preparation, then they can be comminuted by processes known to the person skilled in the art. Preference is given to a two-stage comminution, where the first comminution step is followed by a strand or tape extrusion. The strands or tapes obtained in the operation can be converted into chips or cylindrical pellets in a known manner using chippers or granulators.

For further comminution, known grinding units of all types and designs are suitable. Depending on the desired application or depending on the nature of the cosmetic or pharmaceutical formulation, it is possible to prepare particle sizes with a diameter of from 1 to 1000 μm. Preferred particle sizes are in the range between 1 and 100 μm, particularly preferably between 15 and 50 m.

The thickness of the pigments is between 0.5 and 100 μm, preferably between 1 and 50 μm, particularly preferably between 1.0 and 10 μm.

Very particular preference is given to broadband reflectors which can be produced by mixing two or more cholesteric liquid-crystalline pigments which each have different IR reflection maxima.

Moreover, pigments of different direction of rotation of light can be obtained by mixing two different pigments of the cholesteric material of opposite twist (helicity). Pigments having cholesteric structures of opposite twist are obtainable, for example, by adding in each case the individual enantiomers or diasteromers of the chiral additives to the achiral liquid-crystalline polymerizable monomers. The pitch of the structures of opposite twist may here be identical or different. Pigments with different direction of rotation of light reflect significantly more radiation than pigments with the same direction of rotation of light.

It is also possible to firstly mix the cholesteric components of opposite twist, then to convert these by the abovementioned hardening into the pigments already described, and to use them as IR reflectors in cosmetic and dermatological compositions.

In addition to the abovementioned mixtures of cholesteric liquid-crystalline pigments, it is also possible to produce multilayer pigments whose individual layers contain different, three-dimensionally crosslinked cholesteric liquid-crystalline components. The design of such multilayer pigments can be varied widely. For example, it is possible, inter alia, to apply

individual layers of crosslinked cholesteric material of opposite twist or

individual layers of crosslinked cholesteric liquid-crystalline material of identical twist direction but different pitch and thus different reflection properties over one another.

Details with regard to preparation of such multilayer cholesteric pigments are given in DE-A-197 38 368.

By choosing the composition of the pigments appropriately it is possible to achieve the desired reflection in the infrared wavelength region, without a coloration (in the visible region) being exhibited.

The pigments can be incorporated into the cosmetic and pharmaceutical preparations by simple mixing. This results in no aggregation or separation of the pigment particles.

The light protection agent preparation according to the invention comprises, as constituent B), at least one component which protects against radiation in the ultraviolet ray range from 280 nm to 449 nm. A component which protects against UV radiation is to be understood as meaning a substance (UV filter substance) which absorbs and/or reflects radiation in the ultraviolet ray range. Preferably, constituent B) comprises two or more compounds which absorb and/or reflect in the UV-A and/or UV-B region in order to protect the skin or the hair against the entire range of ultraviolet radiation.

In a preferred embodiment, constituent B) is chosen from the following classes: cholesteric components with a pitch of less than 450 nm, aminobenzoic acids, aminobenzoic esters, bicyclo[2.2.1]heptan-2-ones, hydroxybenzoic esters and hydroxybenzoic acid salts, benzophenones, benzimidazoles, cinnamic esters, 3,3-diphenylacrylic esters, 3-imidazol-4-ylacrylic acids, 3-imidazol-4-ylacrylic esters, alicyclic dienones, triazines, phenylglyoxylic acids and salts, dibenzoylmethanes, 4,4′-diarylbutadienes, benzotriazoles, organosiloxane benzalmalonates, enamines, cyclic iminoesters, inorganic pigments and mixtures thereof.

Suitable cholesteric materials with a pitch of less than 450 nm are cholesteric components corresponding to variants a) to f) of the abovementioned components which reflect in the infrared wavelength region, with the proviso that the pitch of the cholesteric liquid-crystalline phase of component B) is at most 449 nm. Preferred components are those of variants a), b) and c). Particularly preferred cholesteric components of variants a) and b) are described, for example in DE-A-198 24 972, to the entire contents of which reference is hereby made.

Preferred monomers of variant a) comprises at least one chiral liquid-crystalline polymerizable monomer of the formula III,

[Z⁴-Y⁹-(A⁴)ₖ-Y¹⁰-M³-Y¹¹-]₁-X (III)

in which the variables, independently of one another, have the following meanings:

A⁴ is a spacer of chain length from 1 to 30 carbon atoms,

Y⁹ to Y¹¹ is a chemical bond, —O—, —S—, —C(═O)—O—, —O—C(═O)—, —CH═CH—C(═O)—O—, —O—C(═O)—O—, —C(═O)—N(R)— or —(R)N—C(═O)—, —CH₂—O—, —O—CH₂—, —CH═N—, —N═CH—, —N═N—, —(R)N—C(═O)—O— or —O—C(═O)—N(R)—,

M³ is a mesogenic group,

R is hydrogen, C₁-C₄-alkyl,

Z⁴ is hydrogen, C₁-C₄-alkyl, a polymerizable group or a radical which carries a polymerizable group,

X is an n-valent chiral radical,

k is 0 or 1,

l is 1 to 6,

where the radicals Z⁴, Y⁹, Y¹⁰, Y¹¹, A⁴ and M³ may be identical or different and at least one radical Z⁴ represents a polymerizable group or a radical which contains a polymerizable group, if 1 is greater than 1.

Preferred cholesteric mixtures of variant b) suitable as component B) comprise at least one achiral liquid-crystalline monomer and a chiral compound. These differ only with regard to their percentage composition, but not with regard to their material composition, of constituent A) in the embodiment of variant b).

The polymerizable mixture of variant b) preferably comprises at least one achiral liquid-crystalline polymerizable monomer of the formula IV

Z⁵-Y¹³-(A⁵)ₒ-Y¹⁴-M⁴-Y¹⁵-(A⁶)_{q}-Y¹⁶-Z⁶ (IV)

in which the variables, independently of one another, have the following meanings:

A⁵ and A⁶ are a spacer of chain length from 1 to 30 carbon atoms,

M⁴ is a mesogenic group,

Y¹³ to Y¹⁶ is a chemical bond, —O—, —S—, —C(═O)—O—, —O—C(═O)—, —CH═CH—C(═O)—O—, —O—C(═O)—O—, —C(═O)—N(R)— or —(R)N—C(═O)—, —CH₂—O—, —O—CH₂—, —CH═N—, —N═CH—, —N═N—, —(R)N—C(═O)—O— or —O—C(═O)—N(R)—,

R is hydrogen, C₁-C₄-alkyl,

o and q are 0 or 1,

Z⁵ and Z⁶ are hydrogen, C₁-C₄-alkyl, a polymerizable group or a radical which carries a polymerizable group, where at least one of the variables Z⁵ or Z⁶ represents a polymerizable group or a radical which carries a polymerizable group, and

at least one chiral additive.

Numerous compounds are known as chiral dopants for liquid-crystalline phases (e.g. from DE-A 43 42 280 and DE-A 196 11 101, and that stated previously). In addition, the chiral dopants should exhibit good compatibility to the liquid-crystalline compounds, thus enabling effective interaction between these components. The dopant is again added in the amount with which the desired UV reflection is achieved.

Preferred chiral additives are compounds of the formula V

[Z⁴-Y⁹-(A⁴)ₖ-Y¹⁰-M⁵-Y¹¹-]₁-X (V)

in which Z⁴, Y⁹, Y¹⁰, Y¹¹, A⁴, X, k and 1 have the meanings given above and M⁵ is a divalent radical which contains at least one hetero- or isocyclic ring system.

The molecular moiety M⁵ here is similar to the described mesogenic groups since particularly good compatibility with the liquid-crystalline compound is achieved in this way. M⁵ need, however, not be mesogenic since the compound V should bring about a corresponding twisting of the liquid-crystalline phase merely through its chiral structure. Preferred ring systems which are present in M⁵ are the abovementioned structures T, preferred structures M⁵ are those of the abovementioned formula (T-Y⁸)ₛ-T. Further monomers and chiral compounds of group b) are described in WO 97/00600 and DE-A-195 324 08 on which it is based, to the entire contents of which reference is hereby made.

Particularly preferred monomers IV are the following structures:

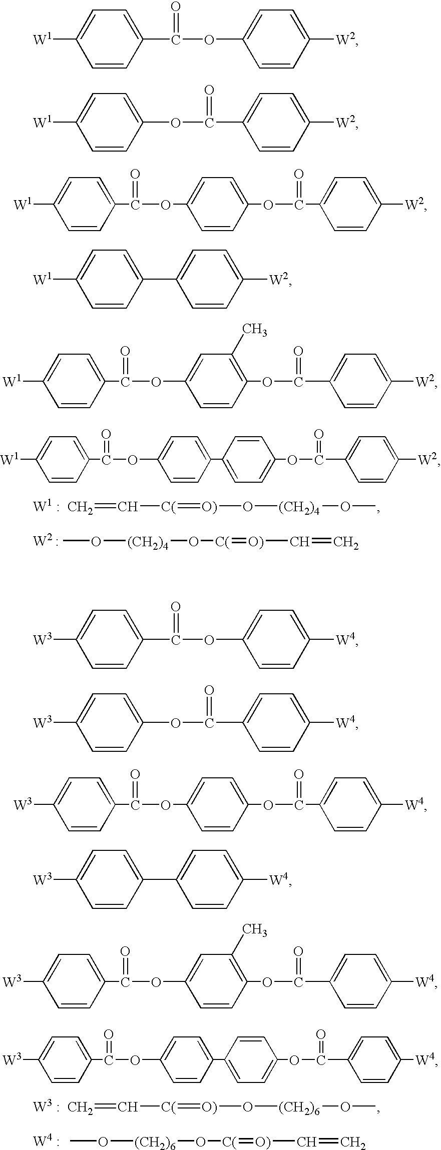

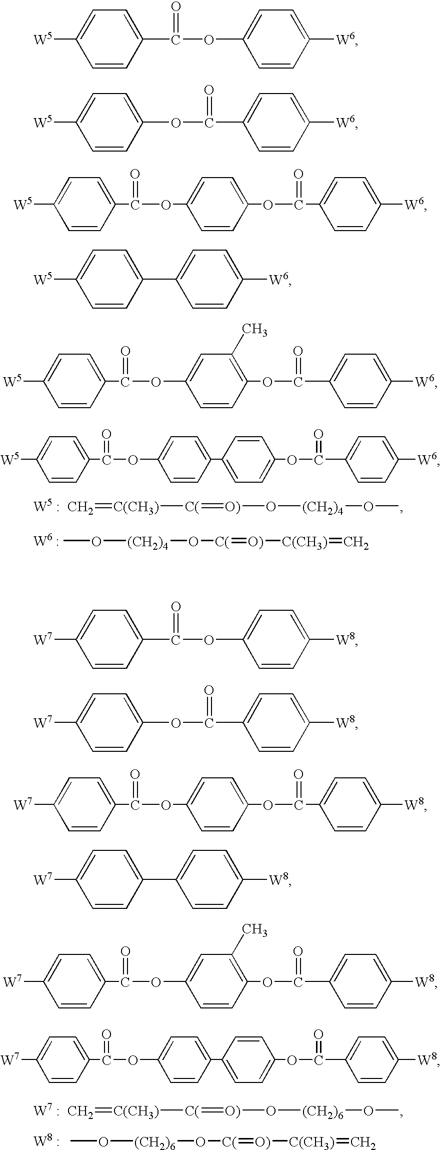

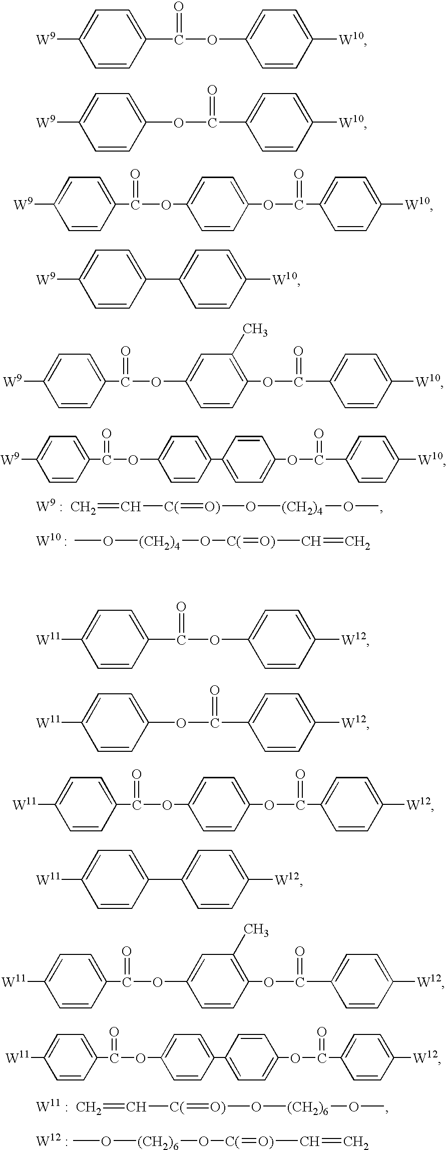

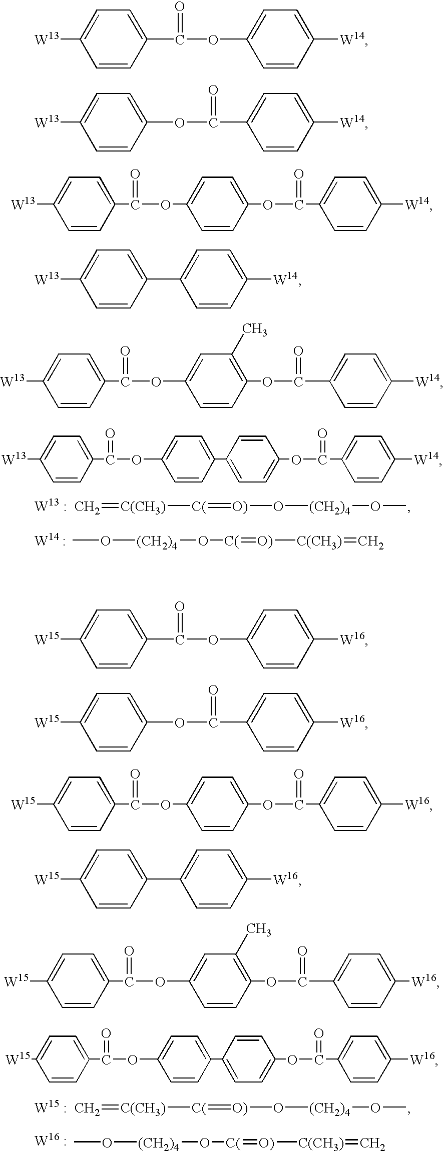

Particularly preferred monomers V are the following structures:

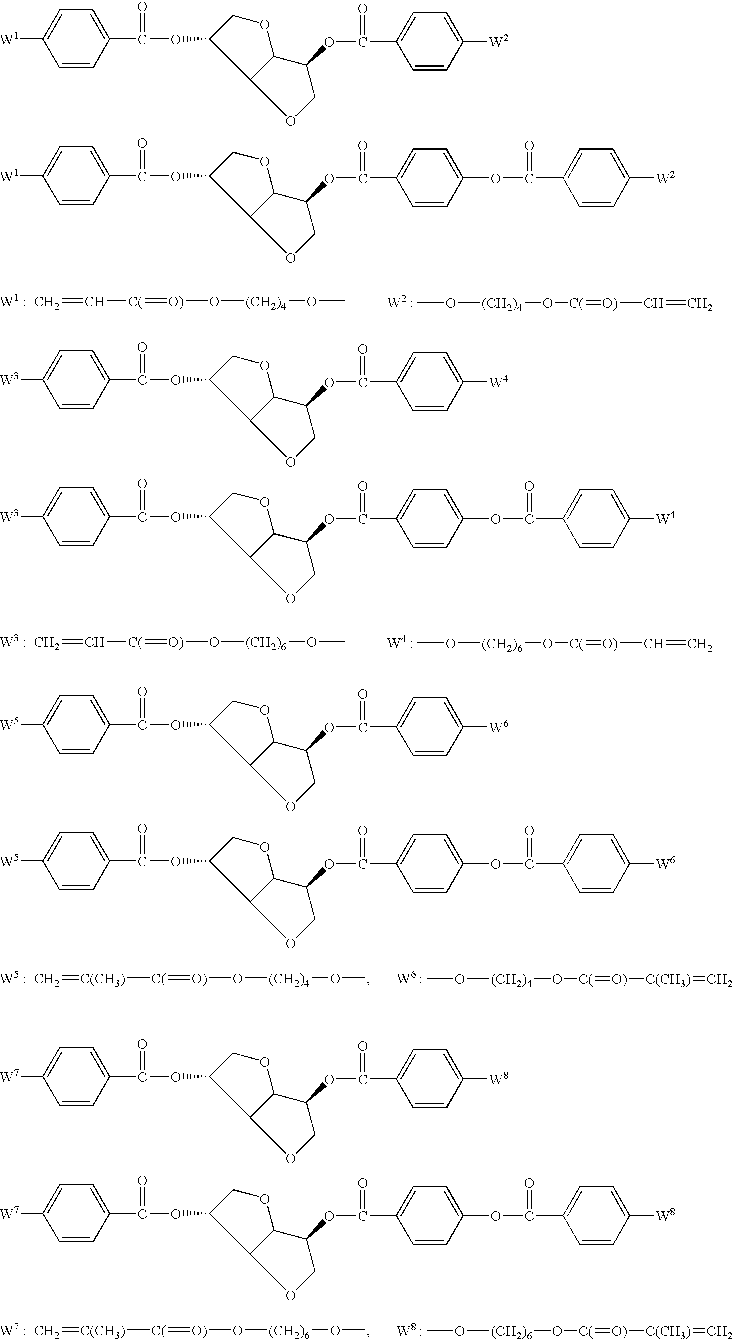

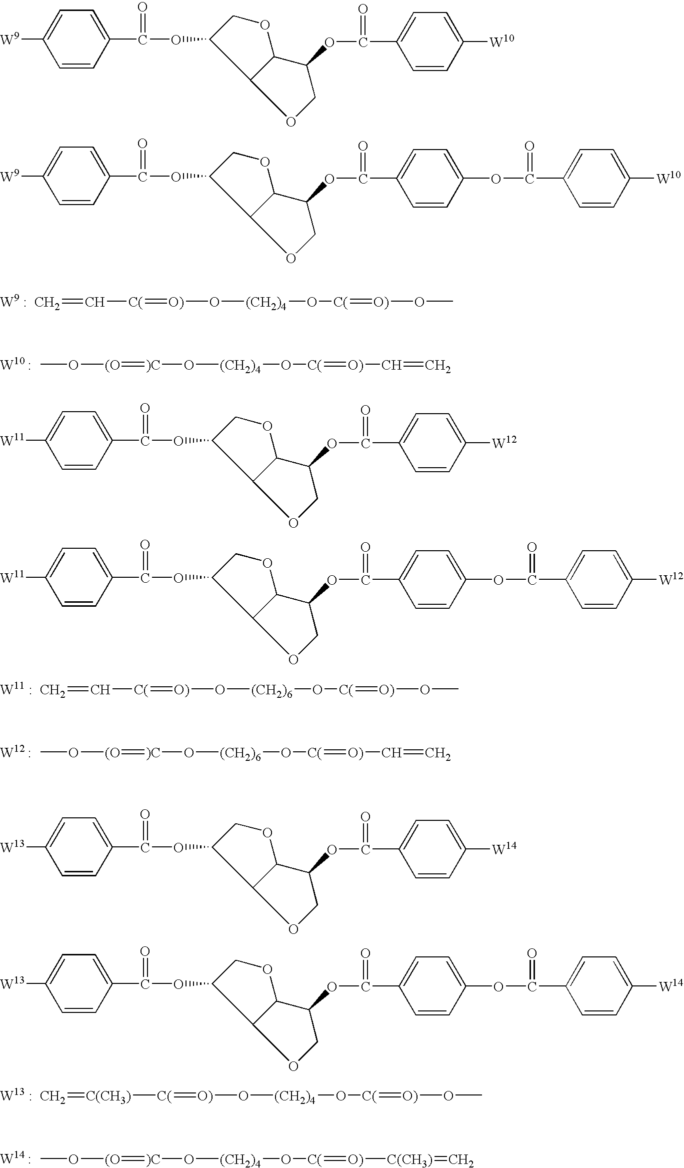

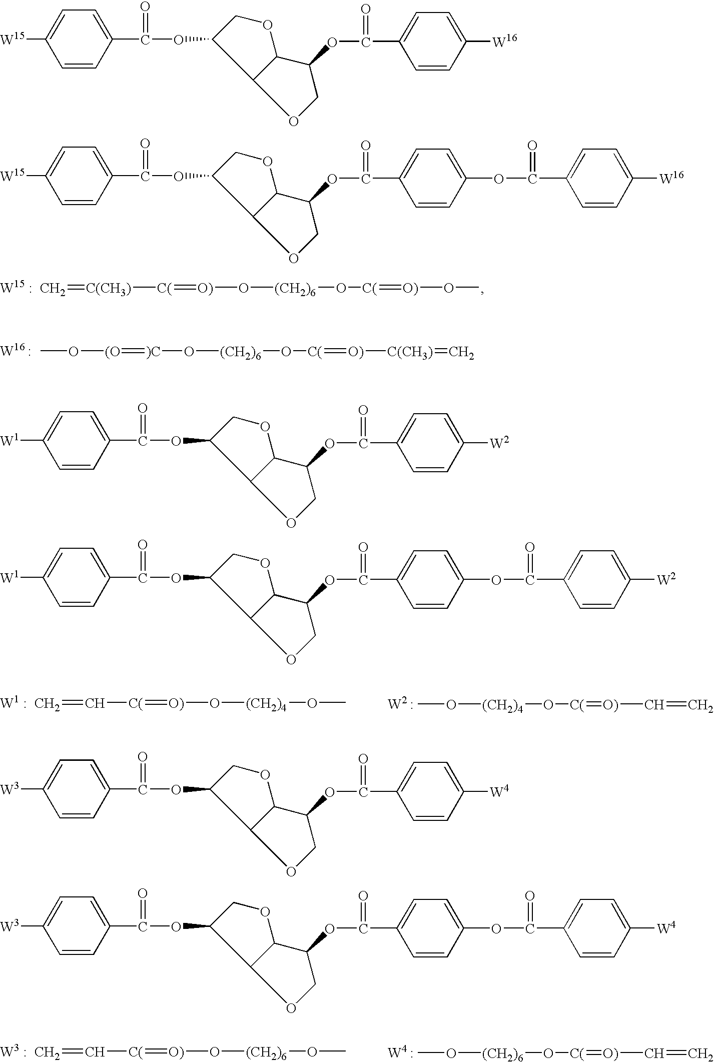

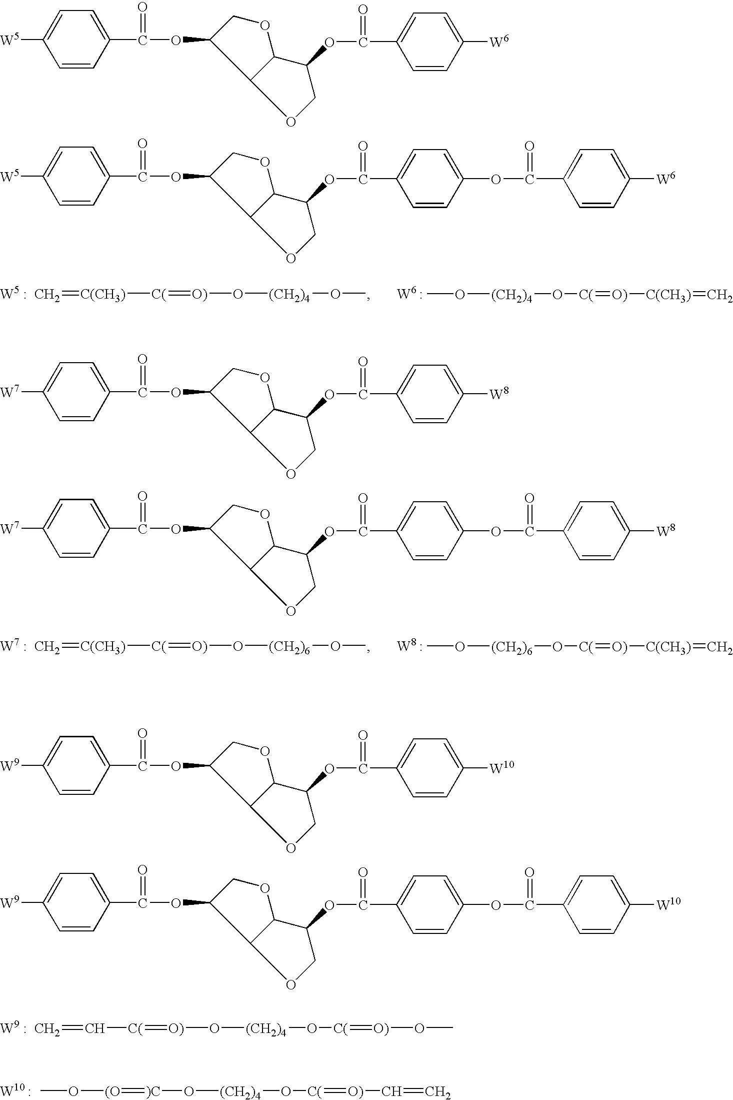

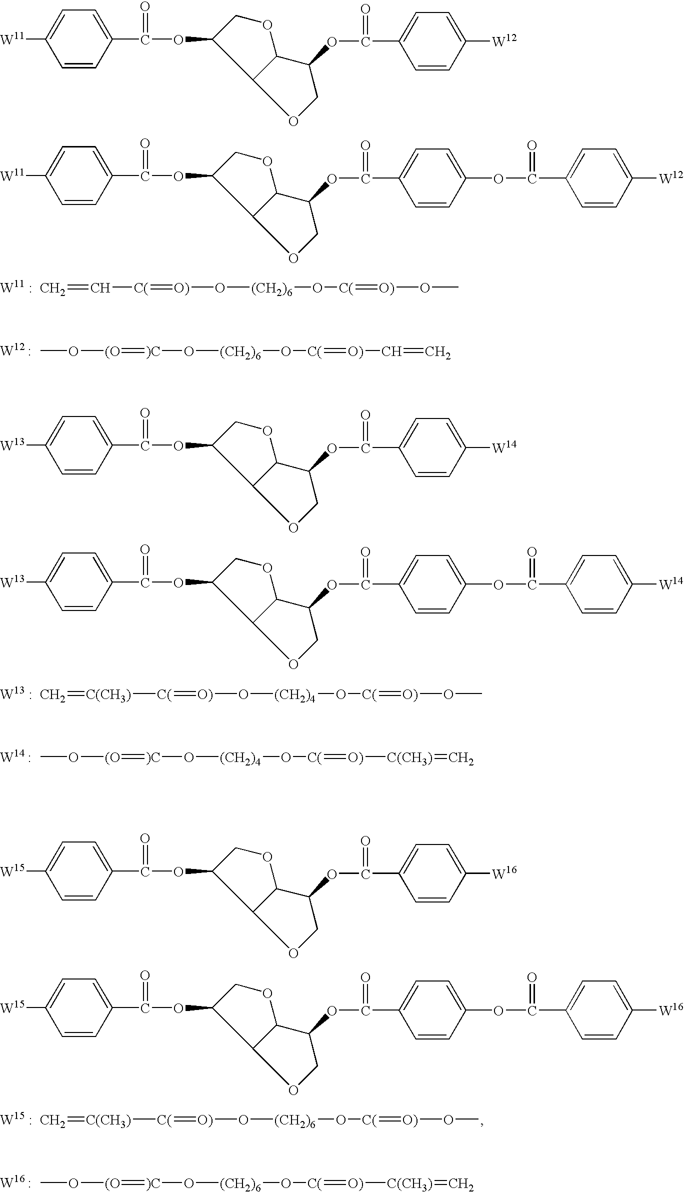

The weight ratios of component IV to component V are in the range from 99:1 to 40:60, preferably in the range from 99:1 to 70:30, particularly preferably from 98:2 to 85:15.

For the preparation of the abovementioned compounds of variants a) and b), reference is made to DE-A-198 24 972.

Preferred polymers of variant c) suitable as component B) are polymers which contain in their polymer chains in an alternating manner the structural element —X—C(═O)—, in which X may be oxygen or NH. Such cholesteric liquid-crystalline polymers are described, for example, in DE-A-198 48 130, to the entire contents of which reference is hereby made. Preferred cholesteric main group polymers are constructed from the following building blocks of the individual monomer groups:

i) 1 to 60 mol %, preferably 3 to 50 mol %, of at least one achiral, bifunctional molecular unit;

ii) 0 to 99 mol %, preferably 5 to 90 mol %, of at least one achiral unit of the group of aromatic hydroxycarboxylic acids, cycloaliphatic hydroxycarboxylic acids, aromatic aminocarboxylic acids and cycloaliphatic aminocarboxylic acids;

iii) 0 to 49.5 mol %, preferably 0 to 40 mol %, of at least one achiral unit from the group of aromatic dicarboxylic acids and cycloaliphatic dicarboxylic acids and

iv) 0 to 99 mol %, preferably 0 to 49.5 mol %, of at least one achiral unit from the group of aromatic diols, cycloaliphatic diols, aromatic diamines and cycloaliphatic diamines,

v) 0 to 5 mol% of a component having more than two functional groups,

where the sum of the individual components i) to v) gives 100 mol %.

The components from group i) are advantageously recruited from the “chiral pool”. In the specialist field (Ullmanns Encycl. Techn., 5th Edition, Vol. A18, p. 183, 1991, VCH-Verlag), this is to be understood as meaning the totality of the naturally occurring chiral compounds. In particular, this includes both the chiral building blocks of animal and also vegetable origin. However, this in no way excludes the use of completely synthetic or partially synthetic chiral molecular building blocks. Thus, valuable chiral components can also be obtained from natural substances by one or more synthetic steps which either do not occur in nature or occur only in small amounts.

In particular, molecular building blocks i) generally means all chiral bifunctional components, for example chiral diols or polyols, chiral dicarboxylic acids, chiral hydroxycarboxylic acids or aminocarboxylic acids.

Particularly preferred chiral, bifunctional monomers are:

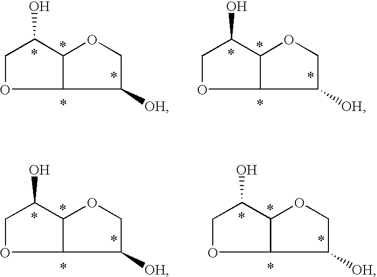

The chiral comonomers are preferably used in an enantiomerically pure form. If enantiomeric mixtures of a comonomer are used, it must be ensured that one enantiomer form is present in an effective excess.

Particularly preferred representatives from the group ii) are:

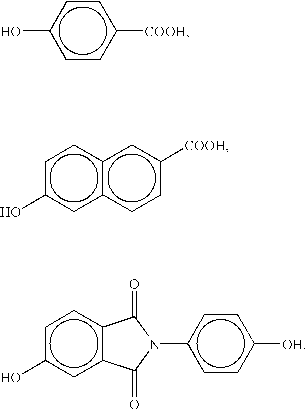

Particularly preferred representatives from the group iii) are:

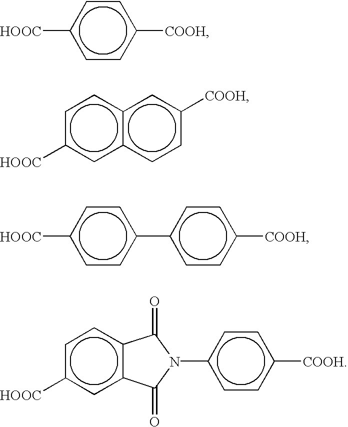

Particularly preferred representatives from the group iv) are:

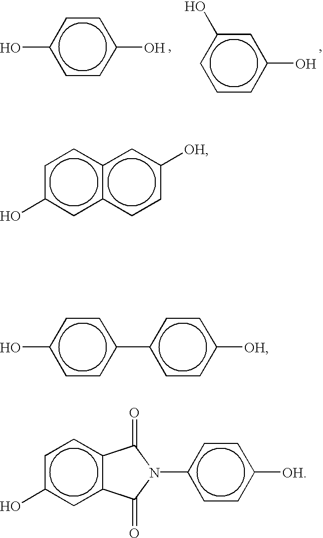

In place of the carboxylic acids, it is also possible to use other carboxylic acid derivatives known to the person skilled in the art, such as, for example, carbonyl chlorides, carboxylic anhydrides or carboxylic esters. In place of the hydroxy components, it is also possible to use corresponding hydroxy derivatives, such as, for example, the acylated hydroxy compounds.

In addition, the polymers can also contain components with more than two functional groups, such as, for example, dihydroxybenzoic acid or trihydroxybenzenes. These components act as branching point within the polymer and are, where appropriate, only added in low concentrations, for example 0 to 5 mol %.

The preparation of these cholesteric polymers is described in DE-A-198 48 130.

Preference is given to using the cholesteric material of constituent B) in the form of pigments. With regard to the preparation of pigments, reference may be made to that stated above (see constituent A)). Details with regard to the preparation of cholesteric liquid-crystalline pigments from cholesteric polymers are given in DE-A-198 48 130.

The suitable and preferred particle sizes and thickness of the pigments are in the range described above for component A).

The preparation according to the invention particularly preferably comprises UV broadband reflectors and UV multilayer reflectors. For the preparation and properties thereof, reference is made to that stated above for component A), and also to DE-A-198 24 972. Preference is given to three-layer pigments in which the two external layers each consist of a hardened cholesteric component and the middle layer can comprise, for example, a binder matrix in which a further UV absorber can be additionally incorporated.

The proportion of cholesteric component in the component which protects against ultraviolet radiation is preferably 10 to 90% by weight, preferably 20 to 70% by weight.

Aminobenzoic acids and aminobenzoic esters suitable as component B) are, for example, p-aminobenzoic acid, 2-amino acid esters, such as 5-methyl-2-(1-methylethyl)-2-aminobenzoate, 4-aminobenzoic esters, where the amino groups may optionally be alkylated or alkoxylated, e.g. 4-NH₂—C₆H₄—C(O)OCH(OH)CH₂OH, 2-ethylhexyl 4-dimethylaminobenzoate, isooctyl 4-dimethylaminobenzoate, 4-N(H₃CCH(OH)CH₂)₂—C₆H₄—C(O)OC₂H₅, polyethoxyethyl 4-bis(polyethoxy)paraaminobenzoate, 1-glyceryl 4-aminobenzoate.

Bicyclo[2.2.1]heptan-2-ones suitable as component B) are e.g. 3,3′-(1,4-phenylenedimethine)bis(7,7-dimethyl-2-oxobicyclo[2.2.1]heptane-1-methanesulfonic acid and its salts, 3-(4′-trimethylammonium)benzylidenebornan-2-one methylsulfate, 3-(4′-sulfo)benzylidenebornan-2-one and its salts, 3-benzylidenebornan-2-one, 3-(4-methylbenzylidene)camphor.

Hydroxybenzoic esters and hydroxybenzoic acid salts suitable as component B) are salicylic esters, such as, for example, 3,3,5-trimethylcyclohexyl salicylate, 4-tert-butylphenyl salicylate, 2-ethylhexyl salicylate, isooctyl salicylate, 4-isopropylbenzyl salicylate, and the triethanolamine salt, and digalloyl trioleate.

Benzophenones suitable as component B) are, for example, 2-hydroxy-4-methoxybenzophenone, 2-hydroxy-4-methoxybenzophenone-5-sulfonic acid and salts thereof, 2,2′-dihydroxy-4-methoxybenzophenone (dioxybenzone), 2-hydroxy-4-methoxy-4-methylbenzophenone (mexenone), 2,2′, 4,4′-tetrahydroxybenzophenone, 2,4-dihydroxybenzophenone, 2,2′-dihydroxy-4,4′-dimethoxybenzophenone-5,5′-disodium sulfonate, benzophenone-11 (mixture of tetrasubstituted benzophenones), 2,2′-dihydroxy-4,4′-dimethoxybenzophenone, compounds of the formula VI,

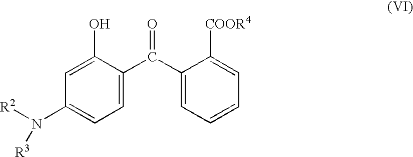

in which R² and R³, independently of one another, are hydrogen, C₁-C₂₀-alkyl, C₃-C₁₀-cycloalkyl or C₃-C₁₀-cycloalkenyl, where the substituents R² and R³ may, together with the nitrogen atom to which they are bonded, form a 5- or 6-membered ring, and R⁴ is C₁-C₂₀-alkyl, C₅-C₆-cycloalkyl.

Benzimidazoles suitable as component B) are, for example, 2-phenylbenzimidazole-5-sulfonic acid and potassium, sodium and triethanolamine salts thereof,

2,2′-(1,4-phenylene)bis-1H-benzimidazole-4,6-disulfonic acid or salts thereof.

Cinnamic esters suitable as component B) are, for example, 2-isoamyl 4-methoxycinnamate, 2-ethylhexyl 4-methoxycinnamate.

3,3-Diphenylacrylic esters suitable as component B) are, for example, those of the formula VII,

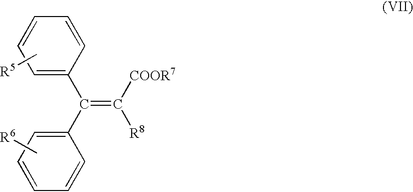

in which R⁵ and R⁶, independently of one another, are hydrogen, linear or branched C₁-C₁₂-alkyl, linear or branched C₁-C₁₂-alkoxy, R⁷ is C₃-C₈-cycloalkyl, which is optionally substituted by C₁-C₄-alkyl groups, in particular methyl, ethyl, n-propyl, 1-methylethyl, n-butyl, 1-methylpropyl, 2-methylpropyl, 1,1-dimethylethyl, and R⁸ is hydrogen or CN, in particular cyclopentyl 2-cyano-3,3-diphenylacrylate, cyclohexyl 2-cyano-3,3-diphenylacrylate, 4-tert-butylcyclohexyl 2-cyano-3,3-diphenylacrylate, ethylhexyl 2-cyano-3, 3-diphenylacrylate.

A suitable 3-imidazol-4-ylacrylic ester is, for example, the ethyl ester.

A phenylglyoxylic acid suitable as component B) is, for example, dimethoxyphenylglyoxylic acid and its sodium salt.

Triazines suitable as component B) are, for example, 2,4,6-triaryl-1,3,5-triazines in which the aryl radicals are optionally substituted, e.g.

2,4,6-trianilino(o-carbo-2′-ethylhexyl-1′-oxy)-1,3,5-triazine, 2,4-bis[4-(2-ethylhexyloxy)-2-hydroxy]phenyl-6-(4-methoxyphenyl)-(1,3,5) triazine, and the other following compounds:

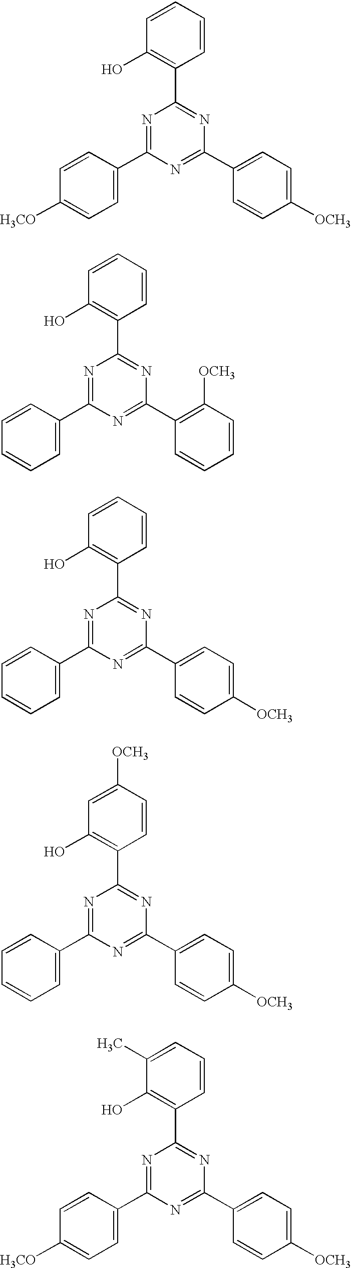

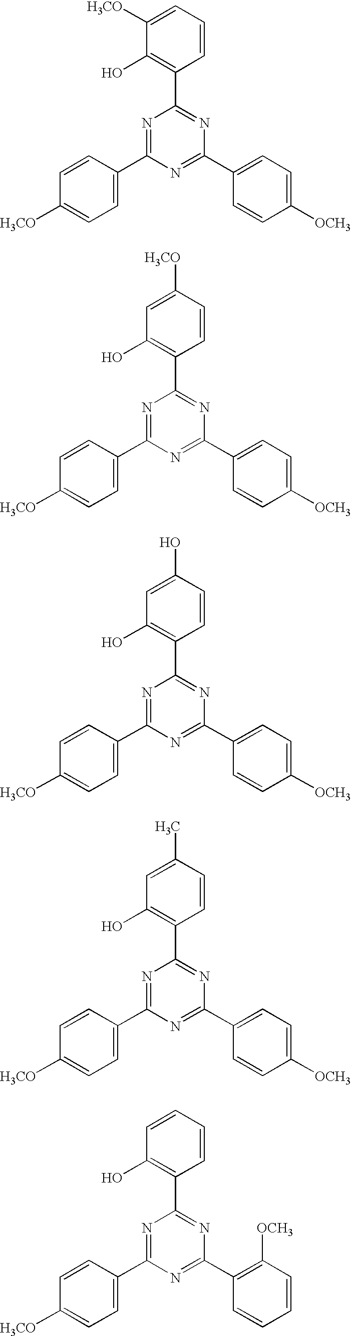

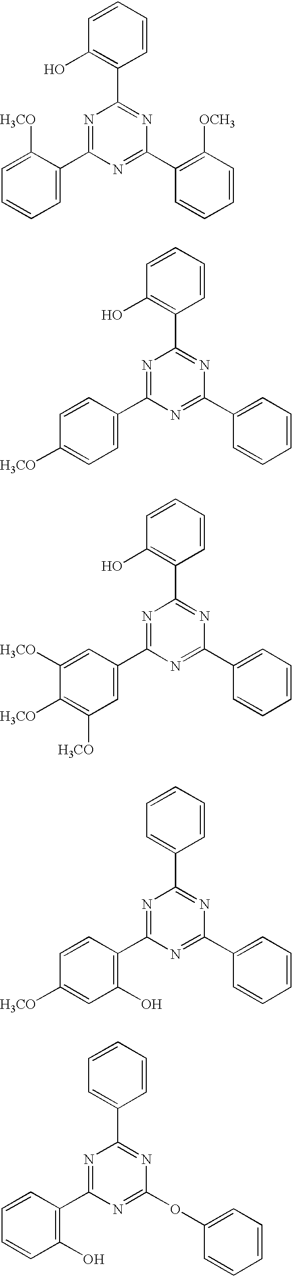

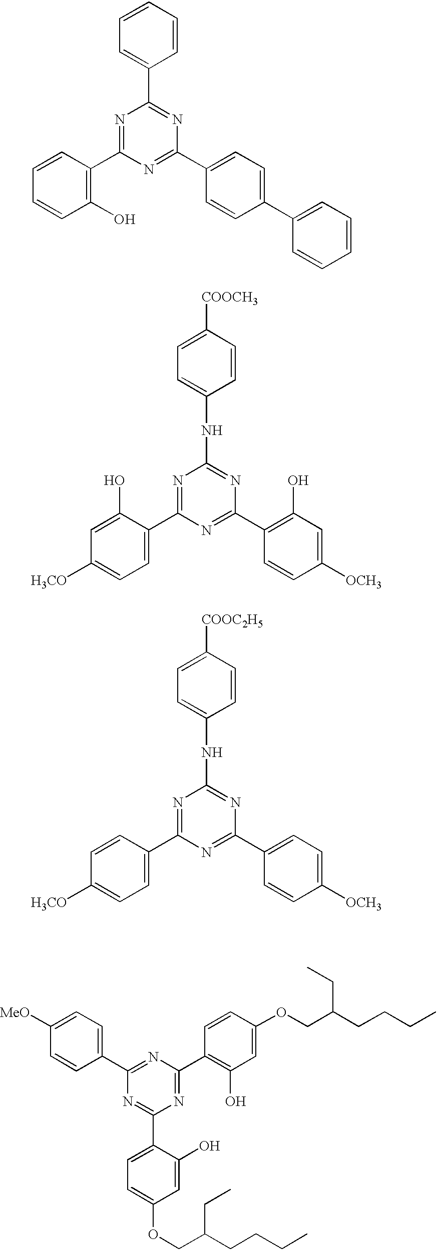

and also triazine derivatives of the formula VIII,

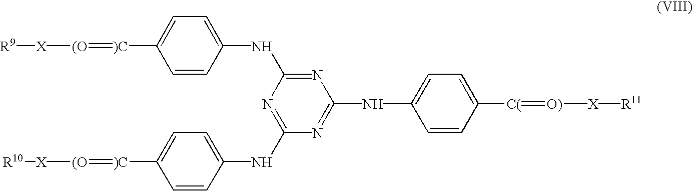

in which R⁹ to R¹¹, independently of one another, are optionally substituted C₁-C₂₀-alkyl, C₅-C₁₀-aryl, C₅-C₁₀-heteroaryl or SpSil, where Sp is a spacer and Sil is a silane, oligosilane or polysiloxane radical

X the divalent radicals

—O— or

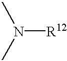

where R¹² is hydrogen or optionally substituted C₁-C₂₀-alkyl, C₅-C₁₀-aryl or C₅-C₁₀-heteroaryl,

preferably

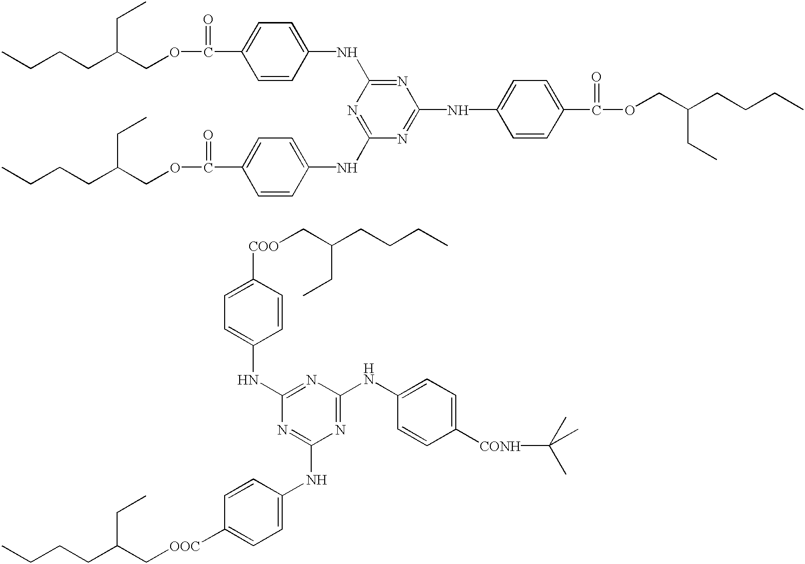

Dibenzoylmethanes suitable as component B) are, for example, 2-methyldibenzoylmethane, 4-methyldibenzoylmethane, 4-isopropyldibenzoylmethane, 4-tert-butyldibenzoylmethane, 2,4-dimethyldibenzoylmethane, 2,5-dimethyldibenzoylmethane, 4,4′-diisopropyldibenzoylmethane, 4-tert-butyl-4′-methoxydibenzoylmethane, 2-methyl-5-isopropyl-4′-methoxydibenzoylmethane, 2-methyl-5-tert-butyl-4′-methoxydibenzoylmethane, 2,4-dimethyl-4′-methoxydibenzoylmethane, 2,6-dimethyl-4-tert-butyl-4′-methoxydibenzoylmethane, in particular 4-tert-butyl-4′-methoxydibenzoylmethane.

4,4′-Diarylbutadienes suitable as component B) are, for example, those of the formula IX,

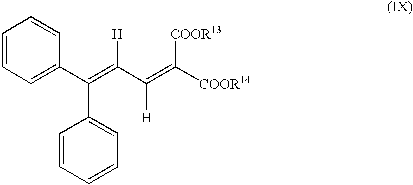

in which R¹³ and R¹⁴, independently of one another, are hydrogen, C₁-C₂₀-alkyl, C₃-C₁₀-cycloalkyl or C₃-C₁₀-cycloalkenyl.

Benzotriazoles suitable as component B) are, for example,

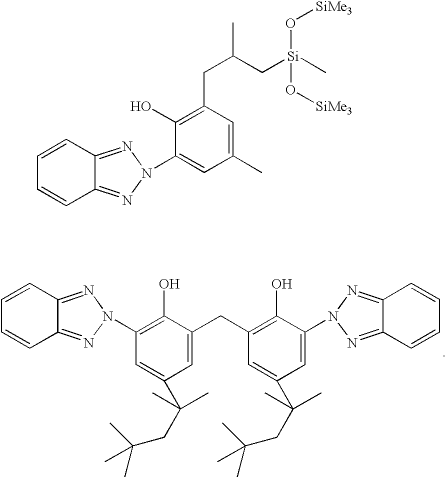

Organosiloxane benzalmalonates suitable as component B) are, for example, those of the formula X,

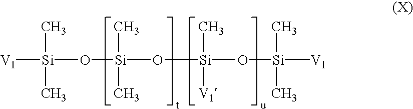

in which

V₁′ is the group

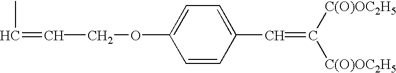

V₁ is a methyl group or V₁′ of the formula XI

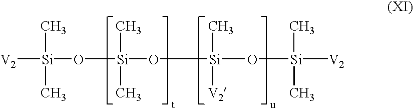

in which V₂′ is the group of the structure

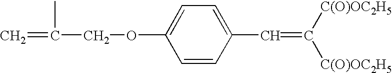

V₂ is a methyl group or V₂′.

An enamine suitable as component B) is, for example,

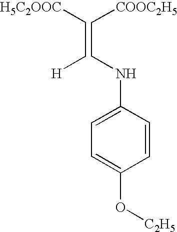

A cyclic iminoester suitable as component B) is, for example,

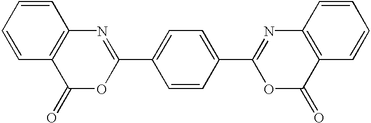

In a preferred embodiment, component B) comprises a light protection agent combination which comprises a hydroxybenzophenone derivative. Suitable combinations are described in DE 100 12 408.9, to which reference is hereby made.

In a further preferred embodiment, component B) comprises 3,3-diphenylacrylates and dibenzoylmethanes. Suitable embodiments are described in DE 100 09 442.2, to which reference is hereby made. Component B also preferably comprises at least one 4-methoxycinnamic ester.

Preferred inorganic pigments suitable as component B) are those 40 based on metal oxides and/or other metal compounds which are insoluble or sparingly soluble in water such as the oxides of titanium (TiO₂), zinc (ZnO), iron (e.g. Fe₂O₃), zirconium (ZrO₂), silicon (SiO₂), manganese (e.g. MnO), aluminum (Al₂O₃), cerium (e.g. Ce₂O₃), mixed oxides of the corresponding metals, and mixtures of such oxides. The pigments are particularly preferably based on titanium dioxide and zinc oxide.

The inorganic pigments are preferably in hydrophobic form, i.e. they are treated superficially to repel water. This superficial treatment may consist in providing the pigments with a thin hydrophobic coat in a manner known per se, as described in DE-A-33 14 742.

Advantageous TiO₂ pigments are obtainable, for example, under the trade names MT 100 T from TAYCA, and also M 160 from Kemira, and T 805 from Degussa.

The cosmetic or dermatological light protection agent preparations advantageously comprise inorganic pigments, in particular micropigments, in amounts of e.g. 0.1% by weight to 30% by weight, preferably in amounts of from 0.5% by weight to 15% by weight, particularly preferably 1% by weight to 10% by weight, very particularly preferably 1.5 to 6% by weight, based on the total weight of the preparations.

The abovementioned compounds of constituent B) may be used individually, as mixtures in each case of one class of compound or generally as mixtures. Finally, it is possible to co-use further substances, known per se, which absorb/reflect in the UV region, provided they are stable in the overall system of the preparation.

The cosmetic and dermatological preparations comprising light protection agents comprise a carrier C) which is chosen from water, water-miscible liquids, hydrophobic components and mixtures. These include water, C₁-C₄-alcohols, such as ethanol and isopropanol, fats, waxes, fatty acids, fatty alcohols, oils, oil-in-water and water-in-oil emulsions, creams and pastes, lip protection stick compositions or fat-free gels.

Suitable emulsions are, inter alia, also O/W macroemulsions, O/W microemulsions or O/W/O emulsions, where the emulsions are obtainable by phase inversion technology, e.g. in accordance with DE-A-197 26 121.

The hydrophobic component (lipid phase) can advantageously be chosen from the following group of substances:

mineral oils, mineral waxes

oils, such as triglycerides of capric acid or of caprylic acid, but preferably castor oil;

fats, waxes and other natural and synthetic fatty substances, preferably esters of fatty acids with alcohols of low carbon number, e.g. isopropanol, propylene glycol or glycerol, or esters of fatty alcohols with alkanoic acids of low carbon number or fatty acids; alkyl benzoate;

silicon oils, such as dimethylpolysiloxanes, diethylpolysiloxanes, diphenylpolysiloxanes, and mixed forms thereof.

The oil phase of the emulsions, oleogels and hydrodispersions or lipodispersions for the purposes of the present invention is advantageously chosen from the group of esters of saturated and/or unsaturated, branched and/or unbranched alkanecarboxylic acids of chain length from 3 to 30 carbon atoms from the group of esters of aromatic carboxylic acids and saturated and/or unsaturated, branched and/or unbranched alcohols of chain length from 3 to 30 carbon atoms. Such ester oils can advantageously be chosen from the group consisting of isopropyl myristate, isopropyl palmitate, isopropyl stearate, isopropyl oleate, n-butyl stearate, N-hexyl laurate, N-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, 2-ethylhexyl laurate, 2-hexyldecyl stearate, 2-octyldodecyl palmitate, oleyl oleate, oleyl erucate, erucyl oleate, erucyl erucate, and synthetic, semisynthetic and natural mixtures of such esters, e.g. jojoba oil.

In addition, the oil phase can advantageously be chosen from the group of branched and unbranched hydrocarbons and hydrocarbon waxes, the silicone oils, the dialkyl ethers, the group of saturated or unsaturated, branched or unbranched alcohols, and the fatty acid triglycerides, namely the triglycerol esters of saturated and/or unsaturated, branched and/or unbranched alkanecarboxylic acids of chain length from 8 to 24, in particular 12 to 18 carbon atoms. The fatty acid triglycerides can, for example, advantageously be chosen from the group of synthetic, semisynthetic and natural oils, e.g. olive oil, sunflower oil, soybean oil, peanut oil, rapeseed oil, almond oil, palm oil, coconut oil, palm kernel oil and the like.

Any mixtures of such oil and wax components can also be used advantageously for the purpose of the present invention. It may in some instances also be advantageous to use waxes, for example, cetyl palmitate, as the sole lipid component of the oil phase.

The oil phase is advantageously chosen from the group consisting of 2-ethylhexyl isostearate, octyldodecanol, isotridecyl isononanoate, isoeicosane, 2-ethylhexyl cocoate, C₁₂-C₁₅-alkylbenzoate, caprylic/capric triglyceride, dicapryl ether.

Mixtures of C₁₂-C₁₅-alkyl benzoate, 2-ethylhexyl isostearate and isotridecyl isononanoate are particularly advantageous.

Of the hydrocarbons, paraffin oil, squalane and squalene are to be used advantageously for the purposes of the present invention.

The oil phase can also advantageously have a content of cyclic or linear silicone oils or consist entirely of such oils, although it is preferred to use an additional content of other oil phase components apart from the silicone oil or the silicone oils.

Cyclomethicone (octamethylcyclotetrasiloxane) is advantageously used as silicone oil to be used according to the invention. However, other silicone oils are also advantageously to be used for the purposes of the invention, for example hexamethylcyclotrisiloxane, polydimethylsiloxane, poly(methylphenylsiloxane).

Also particularly advantageous are mixtures of cyclomethicone and isotridecyl isonononoate, and of cyclomethicone and 2-ethylhexyl isostearate.

The aqueous phase of the preparation according to the invention optionally advantageously comprises:

alcohols, diols or polyols of low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products.

The preparations according to the invention can comprise cosmetic auxiliaries. Customary cosmetic auxiliaries which are suitable as additives are, for example, coemulsifiers, stabilizers, thickeners, biogenic active ingredients, film formers, fragrances, dyes, pearlizing agents, preservatives, pigments, electrolytes (e.g. magnesium sulfate), insect repellents and pH regulators. Suitable coemulsifiers are preferably known W/O and also O/W emulsifiers, such as, for example, polyglycerol esters, sorbitan esters or partially esterified glycerides. Stabilizers which can be used are metal salts of fatty acids, such as, for example, magnesium stearate, aluminum stearate and/or zinc stearate. Suitable thickeners are, for example, crosslinked polyacrylic acids and derivatives thereof, polysaccharides, in particular xanthan gum, guar guar, agar agar, alginates and Tyloses, carboxymethylcellulose and hydroxyethylcellulose, and also fatty alcohols, monoglycerides and fatty acids, polyacrylates, polyvinyl alcohol and polyvinylpyrrolidone. Biogenic active ingredients are to be understood as meaning, for example, plant extracts, protein hydrolysates and vitamin complexes. Customary film formers are, for example, hydrocolloids, such as chitosan, microcrystalline chitosan or quaternized chitosan, polyvinylpyrrolidone, vinylpyrrolidone-vinyl acetate copolymers, polymers of the acrylic acid series, quaternary cellulose derivatives and similar compounds. Examples of suitable preservatives are formaldehyde solution, p-hydroxybenzoate or sorbic acid. Suitable pearlizing agents are, for example, glycol distearic esters, such as ethylene glycol distearate, but also fatty acids and fatty acid monoglycol esters. Dyes which can be used are the substances approved and suitable for cosmetic purposes, as listed, for example, in the publication “Kosmetische Färbemittel” [Cosmetic Colorants] from the Dyes Commission of the German Research Society, published by Verlag Chemie, Weinheim, 1984. These dyes are customarily used in a concentration of from 0.001 to 0.1% by weight, based on the total mixture.

The preparation according to the invention can also additionally comprise at least one insect repellent. These include, for example, 2-ethyl-1,3-hexanediol, 2-ethyl-2,3-hexanediol, 4,5-bis(2-butylene)tetrahydro-2-furaldehyde, dimethyl phthalate, di-n-propyl isocinchomeronate and N,N-diethyl-m-toluamide. It is optionally admixed in an amount up to 15% by weight, preferably 5 to 15% by weight, based on the total weight of the preparation.

An additional content of antioxidants is generally preferred. According to the invention, favorable antioxidants which can be used are all antioxidants customary or suitable for cosmetic and/or dermatological applications.

The antioxidants are advantageously chosen from the group consisting of amino acids (e.g. glycine, histidine, tyrosine, tryptophane) and derivatives thereof, imidazoles (e.g. urocanic acid) and derivatives thereof, peptides, such as D,L-carnosine, D-carnosine, L-carnosine and derivatives thereof (e.g. anserine), carotenoids, carotenes (e.g. β-carotene, lycopene) and derivatives thereof, chlorogenic acid and derivatives thereof, lipoic acid and derivatives thereof (e.g. dihydrolipoic acid), aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof), and salts thereof, dilauryl thiodipropionate, distearyl thiodipropionate, thiodipropionic acid and derivatives thereof (esters, ethers, peptides, lipids, nucleotides, nucleosides and salts) and sulfoximine compounds (e.g. buthionine sulfoximines, homocysteine sulfoximines, buthionine sulfones, penta-, hexa-, heptathionine sulfoximine) in very low tolerated doses (e.g. pmol to μmol/kg), and also (metal)chelating agents (e.g. α-hydroxy fatty acids, palmitic acid, phytic acid, lactoferrine), α-hydroxy acids (e.g. citric acid, lactic acid, malic acid), humic acid, bile acid, bile extracts, bilirubin, biliverdine, EDTA and derivatives thereof, unsaturated fatty acids and derivatives thereof (e.g. γ-linolenic acid, linoleic acid, oleic acid), folic acid and derivatives thereof, ubiquinone and ubiquinole and derivatives thereof, vitamin C and derivatives thereof (e.g. ascorbyl palmitate, Mg ascorbyl phosphate, ascorbyl acetate), tocopherol and derivatives (e.g. vitamin E acetate, tocotrienol), vitamin A and derivatives (vitamin A palmitate), and coniferyl benzoate of benzoin resin, rutinic acid and derivatives thereof, α-glycosylrutin, ferulic acid, furfurylideneglucitol, carnosine, butylhydroxytoluene, butylhydroxyanisole, nordihydroguaiacic acid, nordihydroguaiaretic acid, trihydroxybutyrophenone, uric acid and derivatives thereof, mannose and derivatives thereof, zinc and derivatives thereof (e.g. ZnO, ZnSO₄), selenium and derivatives thereof (e.g. selenomethionine), stilbenes and derivatives thereof (e.g. stilbene oxide, trans-stilbene oxide).

The amount of the abovementioned antioxidants (one or more compounds) in the preparations is preferably 0.001 to 30% by weight, particularly preferably 0.05 to 20% by weight, in particular 1 to 10% by weight, based on the total weight of the preparation.

If vitamin E and/or derivatives thereof are the antioxidant(s), it is advantageous to choose the respective concentrations thereof from the range from 0.001-10% by weight, based on the total weight of the formulation. If vitamin A or vitamin A derivatives or carotenes or derivatives thereof are the antioxidant(s), it is advantageous to choose the respective concentrations thereof from the range 0.001-10% by weight, based on the total weight of the formulation.

The preparation can be prepared in a manner known per se, i.e. for example by hot, cold, hot-hot/cold or PIT emulsification. This is a purely mechanical process, no chemical reaction takes place.

The light protection agent preparation according to the invention generally comprises

1 to 20% by weight, preferably 7 to 15% by weight, of component A),

1 to 35% by weight, preferably 5 to 25% by weight of component B) and

carrier C) and auxiliaries to 100% by weight.

The formulation base of dermatological formulations according to the invention preferably comprises pharmaceutically acceptable auxiliaries. The auxiliaries which are known for use in the pharmaceutical sector, food technology and related fields, in particular those listed in relevant pharmacopoeia (e.g. DAB Ph. Eur. BP NF), and other auxiliaries whose properties do not oppose a physiological application are pharmaceutically acceptable.

Suitable auxiliaries may be: lubricants, wetting agents, emulsifying and suspending agents, preservatives, antioxidants, antiirritatives, chelating agents, emulsion stabilizers, film formers, gel formers, odor masking agents, resins, hydrocolloids, solvents, solubility promoters, neutralizing agents, permeation accelerators, pigments, quaternary ammonium compounds, refatting and superfatting agents, ointment, cream or oil bases, silicone derivatives, stabilizers, sterilizing agents, propellants, drying agents, opacifiers, thickeners, waxes, emollients, white oils. A formulation with regard to this is based on specialist knowledge, as given, for example, in Fiedler, H. P. Lexikon der Hilfsstoffe für Pharmazie, Kosmetik und angrenzende Gebiete [Lexikon of auxiliaries for pharmacy, cosmetics and related fields], 4th Edition, Aulendorf: ECV-Editio-Kantor-Verlag, 1996.

Examples of suitable dermatological formulations are ointments, creams, hydrogels, pastes, and liquid medicament forms, such as solutions, emulsions, in particular oil-in-water emulsions, suspensions, for example lotions etc. If desired, it is also possible to use liposomes or microspheres.

To prepare the dermatological preparations according to the invention, active ingredients can be mixed or diluted with a suitable auxiliary (excipient). Excipients may be solid, semisolid or liquid materials which may serve as a vehicle, carrier or medium for the active ingredient. The admixing of further auxiliaries takes place, if desired, in the manner known to the person skilled in the art.

If desired, two or more active ingredient components can be formulated together. They may, however, also be firstly processed separately and then combined in a suitable medicament form.

For use, the cosmetic or dermatological preparations according to the invention are applied to the skin and/or the hair in an adequate amount in the manner customary for cosmetics.

The cosmetic and/or dermatological light protection formulations according to the invention can be used for cosmetic and/or dermatological light protection, and also for the treatment, the care and the cleansing of the skin and/or the hair and as a make-up product in decorative cosmetics.

Particular preference is given to those cosmetic and dermatological preparations which are in the form of a sunscreen.

Such sunscreen preparations may be in liquid, paste or solid form, for example in the form of water-in-oil creams, oil-in-water creams and lotions, aerosol foam creams, gels, oils wax pencils, powders, sprays, for example in the form of a spray oil or spray emulsion or alcoholic aqueous lotions.

Suitable propellants for aerosols are the customary propellants, for example propane, butane, pentane, dimethyl ether and others.

To protect human hair against UV rays, the preparations to be used according to the invention can be incorporated into shampoos, lotions, gels, hairspray, hair lacquer, shine spray, hairstyling products, aerosol foam creams, conditioners, setting lotions, blow-drying lotions, blow-drying setting compositions or emulsions. The respective formulations can be used here, inter alia, for the washing, coloring and styling of hair.

The preparations according to the invention are highly suitable for protecting the human skin or the human hair against thermal radiation in the infrared wavelength range from 750 to 2500 nm and against UV radiation in the range from 280 to 450 nm. The warming of the skin or hair treated with the preparation according to the invention is significantly less than that for skin and hair which is untreated or treated only with UV light protection agent. The reduction in thermal radiation is at least 20%, preferably at least 30%. The thermal radiation in the infrared region of the electromagnetic spectrum is for the most part reflected and not absorbed, meaning that skin and hair do not heat up as a result of thermal radiation. The preparations according to the invention are characterized by a pleasant feel on the skin. A fresher feel is imparted. Above 750 nm, in particular in the wavelength range from 751 to 1600 nm, at least 60%, in particular 70% and very particularly preferably at least 80%, of the incident radiation are reflected.

The examples below serve to illustrate the invention in more detail without limiting it thereto.

I. Preparation

To prepare the pigments 1 and 2, the achiral, nematic monomer used was a compound of the formula 4, and the chiral dopant used was the compound of the formula 2B₁ or 2B₂. The helical superstructure of the pigments comprising the compound 2B₁ was right-handed, and the helical superstructure of the pigments comprising the compound 2B₂ was left-handed.

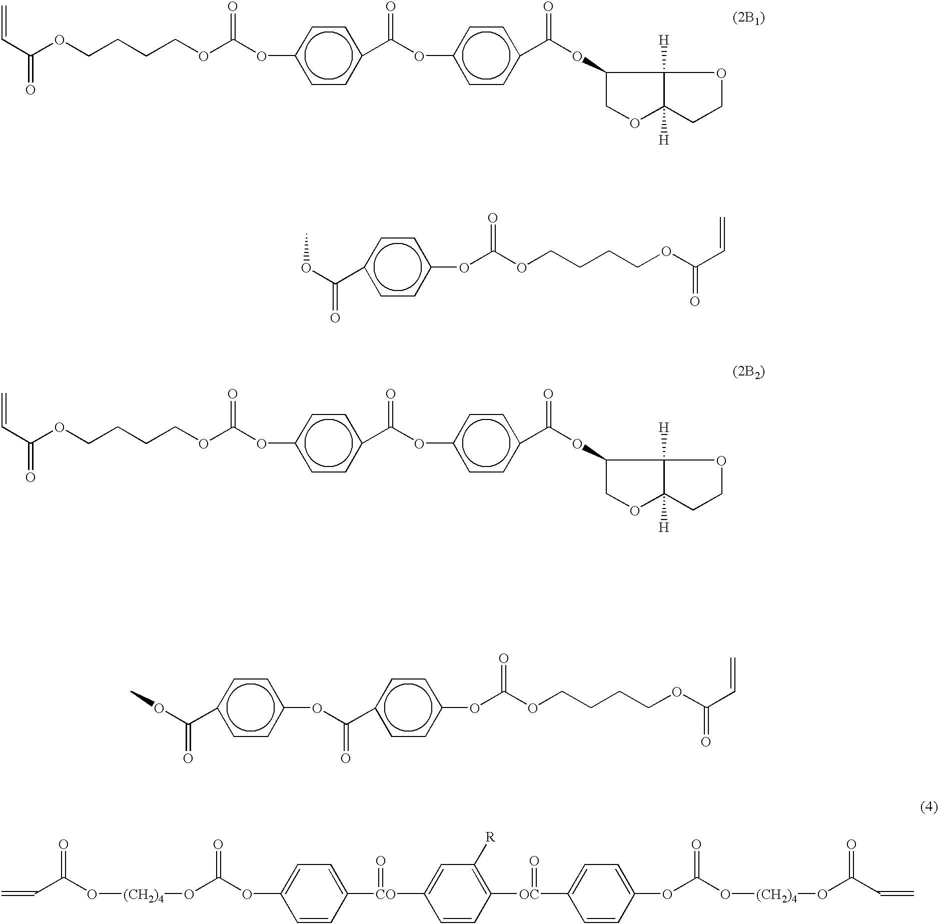

Pigment 1: Reflection Range 750-1200 nm

To prepare the pigment 1, eight cholesteric liquid-crystalline mixtures were used whose compositions and also the wavelength of the reflection maximum max of each individual mixture can be found in Table 1 below.

TABLE 1

| Formula 2B₁ | Formula 2B₂ | Formula 4 | |
|---|---|---|---|
| [% by wt.] | [% by wt.] | [% by wt.] | λₘₐₓ [nm] |
| 2.85 | — | 97.15 | 850 |
| 2.45 | — | 97.55 | 975 |
| 1.92 | — | 98.08 | 1200 |
| 1.65 | — | 98.35 | 1560 |
| — | 7.60 | 92.40 | 850 |
| — | 6.55 | 93.45 | 975 |
| — | 5.10 | 94.90 | 1200 |
| — | 4.10 | 95.90 | 1560 |

For application, the individual mixtures were each dissolved in methyl ethyl ketone. Using a doctor blade, the eight mixtures were applied to a polyethylene terephthalate film individually one after the other on top of one another with a moist film thickness of in each case 2.5 μm, the solvent was stripped off at 70° C. and the resulting film was crosslinked and hardened by UV irradiation. Coating was carried out in accordance with a method described in DE-A 196 38 797. The helical superstructure of four layers was right-handed, that of the other four layers was left-handed. The hardened multicholesteric layer was removed from the carrier and classified by grinding and subsequent screening. The particle size of the pigment particles was in the range<50 μm.

Pigment 2: Reflection Range 850-1600 nm

To prepare pigment 2, six cholesteric mixtures were used, the compositions of which, and the wavelength of the reflection maximum λₘₐₓ of each mixture can be found in Table 2.

TABLE 2

| Formula 2B₁ | Formula 2B₂ | Formula 4 | |
|---|---|---|---|
| [% by wt.] | [% by wt.] | [% by wt.] | λₘₐₓ [nm] |
| 2.45 | — | 97.55 | 975 |
| 1.92 | — | 98.08 | 1200 |
| 1.65 | — | 98.35 | 1560 |
| — | 6.55 | 93.45 | 975 |
| — | 5.10 | 94.90 | 1200 |
| — | 4.10 | 95.90 | 1560 |

Pigment 2 was prepared in a manner analogous to that for Pigment 1. The helical superstructure of three layers was right-handed, the other three layers were left-handed. Each layer was then comminuted and the pigments were mixed.

II. Preparations

### EXAMPLE 1

Lipcare composition

| Content by mass | |
|---|---|
| (% by wt.) | |
| ad 100 | Eucerinum anhydricum |
| 10.00 | Glycerol |
| 10.00 | Titanium dioxide, micronized |
| 3.00 | Ethylhexyl 2-cyano-3,3-diphenylacrylate |
| 2.00 | 4-tert-Butyl-4′-methoxydibenzoylmethane |
| 8.00 | IR pigment 1 |
| 8.00 | Octyl methoxycinnamate |
| 5.00 | Zinc oxide |
| 4.00 | Castor oil |
| 4.00 | Pentaerythrityl stearate/caprate/caprylate/adipate |
| 3.00 | Glyceryl stearate SE |
| 2.00 | Beeswax |
| 2.00 | Microcrystalline wax |
| 2.00 | Quaternium-18 bentonite |
| 1.50 | PEG-45/dodecyl glycol copolymer |

### EXAMPLE 2

Composition for sunblock containing micropigments

| Content by mass | |
|---|---|
| (% by wt.) | |
| ad 100 | Water |
| 10.00 | Octyl methoxycinnamate |
| 6.00 | PEG-7 hydrogenated castor oil |
| 6.00 | Titanium dioxide, micronized |
| 3.00 | Ethylhexyl 2-cyano-3,3-diphenylacrylate |
| 1.50 | 4-tert-Butyl-4′-methoxydibenzoylmethane |
| 10.00 | IR pigment 2 |
| 5.00 | Mineral oil |
| 5.00 | Isoamyl p-methoxycinnamate |
| 5.00 | Propylene glycol |
| 3.00 | Jojoba oil |
| 3.00 | 4-Methylbenzylidenecamphor |
| 2.00 | PEG-45/dodecyl glycol copolymer |
| 1.00 | Dimethicone |
| 0.50 | PEG-40 hydrogenated castor oil |
| 0.50 | Tocopheryl acetate |
| 0.50 | Phenoxyethanol |
| 0.20 | EDTA |

### EXAMPLE 3

Grease-free gel

| Content by mass | |
|---|---|
| (% by wt.) | |
| ad 100 | Water |
| 8.00 | Octyl methoxycinnamate |
| 7.00 | Titanium dioxide, micronized |
| 3.00 | Ethylhexyl 2-cyano-3,3-diphenylacrylate |
| 1.50 | 4-tert-Butyl-4′-methoxydibenzoylmethane |
| 13.00 | IR pigment 2 |
| 5.00 | Glycerol |
| 5.00 | PEG-25 PABA |
| 1.00 | 4-Methylbenzylidenecamphor |
| 0.40 | Acrylates C₁₀-C₃₀ alkyl acrylate crosspolymer |
| 0.30 | Imidazolidinylurea |
| 0.25 | Hydroxyethylcellulose |
| 0.25 | Sodium methylparaben |
| 0.20 | Disodium EDTA |
| 0.15 | Fragrance |
| 0.15 | Sodium propylparaben |
| 0.10 | Sodium hydroxide |

### EXAMPLE 4

Suncream

| Content by mass | |
|---|---|
| (% by wt.) | |
| ad 100 | Water |
| 8.00 | Octyl methoxycinnamate |
| 8.00 | Titanium dioxide, micronized |
| 6.00 | PEG-7 hydrogenated castor oil |
| 3.00 | Ethylhexyl 2-cyano-3,3-diphenylacrylate |
| 1.50 | 4-tert-Butyl-4′-methoxydibenzoylmethane |
| 10.00 | IR pigment 1 |
| 6.00 | Mineral oil |
| 5.00 | Zinc oxide |
| 5.00 | Isopropyl palmitate |
| 0.30 | Imidazolidinylurea |
| 3.00 | Jojoba oil |
| 2.00 | PEG-45/dodecyl glycol copolymer |
| 1.00 | 4-Methylbenzylidenecamphor |
| 0.60 | Magnesium stearate |
| 0.50 | Tocopheryl acetate |
| 0.25 | Methylparaben |
| 0.20 | Disodium EDTA |
| 0.15 | Propylparaben |

### EXAMPLE 5

Suncream, water-resistant

| Content by mass | |
|---|---|
| (% by wt.) | |
| ad 100 | Water |
| 8.00 | Octyl methoxycinnamate |
| 5.00 | PEG-7 hydrogenated castor oil |
| 5.00 | Propylene glycol |
| 4.00 | Isopropyl palmitate |
| 4.00 | Caprylic/capric triglyceride |
| 3.00 | Ethylhexyl 2-cyano-3,3-diphenylacrylate |
| 1.50 | 4-tert-Butyl-4′-methoxydibenzoylmethane |
| 20.00 | IR pigment 2 |
| 4.00 | Glycerol |
| 3.00 | Jojoba oil |
| 2.00 | 4-Methylbenzylidenecamphor |
| 2.00 | Titanium dioxide, micronized |
| 1.50 | PEG-45/dodecyl glycol copolymer |
| 1.50 | Dimethicone |
| 0.70 | Magnesium sulfate |
| 0.50 | Magnesium stearate |
| 0.15 | Fragrance |

### EXAMPLE 6

Day lotion with UV protection

| Content by mass | |
|---|---|
| (% by wt.) | |
| ad 100 | Water |
| 2.00 | Cetearyl alcohol |
| 1.00 | Glycerol monostearate |
| 2.00 | Vaseline |
| 7.50 | Octyl methoxycinnamate |
| 4.00 | Octyl salicylate |
| 3.00 | Ethylhexyl 2-cyano-3,3-diphenylacrylate |
| 1.50 | 4-tert-Butyl-4′-methoxydibenzoylmethane |
| 11.00 | Pigment 1 |
| 0.50 | Dimethicone |
| 5.00 | Propylene glycol |
| 0.20 | EDTA |
| 0.20 | Carbomer |
| 5.00 | C₁₂-C₁₅ alkyl benzoate |
| 0.27 | Triethanolamine |
| 1.00 | Tocopheryl acetate |
| q.s. | Fragrance |

### EXAMPLE 7

Day cream with UV protection

| Content by mass | |
|---|---|
| (% by wt.) | |
| ad 100 | Water |
| 2.00 | Cetearyl alcohol |
| 2.00 | Cetyl alcohol |
| 1.00 | Glycerol monostearate |
| 2.00 | Vaseline |
| 7.50 | Octyl methoxycinnamate |
| 4.00 | Octyl salicylate |
| 3.00 | Ethylhexyl 2-cyano-3,3-diphenylacrylate |
| 1.50 | 4-tert-Butyl-4′-methoxydibenzoylmethane |
| 15.00 | Pigment 2 |
| 4.00 | Propylene glycol |
| 0.20 | EDTA |
| 0.20 | Carbomer |
| 0.20 | Xanthan |
| 0.20 | C₁₀-C₃₀ alkyl acrylates crosspolymer |
| 5.00 | C₁₂-C₁₅ alkyl benzoate |
| 0.54 | Triethanolamine |
| 1.00 | Tocopheryl acetate |
| q.s. | Fragrance |
| q.s. | Preservative |

### EXAMPLE 8

Liquid make-up

| Content by mass | |
|---|---|
| (% by wt.) | |
| ad 100 | Water |
| 2.00 | Cetearyl alcohol |
| 2.00 | Ceteareth 25 |
| 6.00 | Glycerol monostearate |
| 1.00 | Cetyl alcohol |
| 8.00 | Paraffin oil |
| 7.00 | Cetearyl octanoate |
| 0.2 | Dimethicone |
| 3.00 | Propylene glycol |
| 1.00 | Panthenol |
| 3.00 | Ethylhexyl 2-cyano-3,3-diphenylacrylate |
| 1.50 | 4-tert-Butyl-4′-methoxydibenzoylmethane |
| 8.00 | Pigment 2 |
| 3.50 | Octyl methoxycinnamate |
| 0.1 | Bisabolol |
| 5.70 | Titanium dioxide |
| 1.10 | Iron oxide |
| q.s. | Fragrance |

### EXAMPLE 9

Hair gel with sun protection

| Content by mass | |
|---|---|
| (% by wt.) | |
| ad 100 | Water |
| 1.20 | Carbomer |
| 0.50 | Hydroxyethylcellulose |
| 4.00 | Triethanolamine |
| 0.70 | PEG-40 hydrogenated castor oil |
| 1.50 | Ethylhexyl 2-cyano-3,3-diphenylacrylate |
| 0.70 | 4-tert-Butyl-4′-methoxydibenzoylmethane |
| 7.00 | Pigment 1 |
| 2.80 | Octyl methoxycinnamate |
| 5.00 | Propylene glycol |
| 0.01 | EDTA |
| q.s. | Fragrance |
| q.s. | Sicovit Patent Blue 85 E 131 |

III. Testing the Cooling Effect

III.1 Preparation Comprising Pigment 1

12 mg of the inventive preparation from Example 4 were applied to a 6 cm² test field on the back of a subject. The area of skin was then irradiated from a distance of 4 cm using a OSRAM Longlife 64623 EVA 12V 100W lamp. The irradiated area corresponded to a circle with a diameter of from 2 to 3 cm. The surface temperature of the skin was immediately ascertained using a “Ranger” AMIR 7814 pyrometer from Ahlborn.

To assess the cooling effect, the experiment was repeated using a sunscreen preparation which, apart from the IR reflectors, had the same composition. In addition, the temperature increase on untreated skin was determined. The results are given in Table 3 below.

TABLE 3

| | | | Skin after |
|---|---|---|---|
| | | Skin after | treatment with |
| | | treatment with | preparation ac- |
| | | UV light protec- | cording to the |
| | Skin [° C.] | tion agent [° C.] | invention [° C.] |
| Without exposure | 32.3 | 32.3 | 32.3 |
| After 5 min, | 32.8 | 32.8 | 32.3 |
| without exposure | | | |
| After exposure | 40.7 | 40.5 | 36.3 |
| for 3 min | | | |
| Δ T | 7.9 | 7.7 | 4.0 |

The test field provided with the preparation according to the invention exhibits a significantly lower temperature increase. It is almost 50% below the temperature increase of untreated skin. In the reflection range from 750 to 1200 nm, a reflection intensity of 90% is observed.

III.2 Preparation Comprising Pigment 2

The test for the cooling effect of pigment 2 was carried out in the manner described in III.1, except that instead of the inventive preparation from Example 4 being applied, 12 mg of the inventive preparation from Example 2 were applied.

| | | Skin after | Skin after |
|---|---|---|---|
| | | treatment with | treatment with |
| | | UV light | preparation ac- |
| | Skin | protection | cording to the |
| | [° C.] | agent [° C.] | invention [° C.] |
| Without exposure | 32.3 | 32.3 | 32.3 |
| After 5 min, | 32.8 | 32.8 | 32.3 |
| without exposure | | | |
| After exposure | 40.7 | 40.4 | 37.6 |
| for 3 min | | | |
| Δ T | 7.9 | 7.6 | 5.3 |

The test field provided with the preparation according to the invention exhibits a significantly lower temperature increase, which is 33% below the temperature increase of untreated skin. In the reflection range from 850 to 1600 nm, a reflection intensity of 85% is observed.

We claim:

1. A cosmetic or dermatological preparation comprising
A) at least one cholesteric component which reflects in the infrared wavelength range from 750 nm to 2500 nm,
B) at least one component which protects against radiation in the ultraviolet ray range from 280 nm to 449 nm and
C) a cosmetically or dermatologically acceptable carrier.

2. A preparation as claimed in claim 1, wherein component A) is chosen from
a) at least one cholesteric polymerizable monomer;
b) at least one achiral, nematic, polymerizable monomer and a chiral compound;
c) at least one cholesteric crosslinkable polymer;
d) at least one cholesteric polymer in a polymerizable diluent or a mixture of polymerizable diluents;
e) at least one cholesteric polymer whose cholesteric phase can be frozen by rapid cooling to below the glass transition temperature; or
f) at least one achiral, liquid-crystalline crosslinkable polymer and a chiral compound;
in a hardened state.

3. A preparation as claimed in claim 1, where component B) is chosen from the following classes: cholesteric components with a pitch of less than 450 nm, aminobenzoic acids, aminobenzoic esters, bicyclo[2.2.1]heptan-2-ones, hydroxybenzoic esters and hydroxybenzoic acid salts, benzophenones, benzimidazoles, cinnamic esters, 3,3-diphenylacrylic esters, 3-imidazol-4-ylacrylic acids, 3-imidazol-4-ylacrylic esters, alicyclic dienones, triazines, phenylglyoxylic acids and salts, dibenzoylmethanes, 4,4′-diarylbutadienes, benzotriazoles, organosiloxane benzalmalonates, enamines, cyclic iminoesters, inorganic pigments and mixtures thereof.

4. A preparation as claimed in claim 3, where the inorganic pigment is chosen from zinc oxide, titanium oxide and mixtures thereof.

5. A preparation as claimed in claim 3, where component B) comprises at least one 3,3-diphenylacrylate and at least one dibenzoylmethane derivative.

6. A preparation as claimed in claim 3, where component B) comprises at least one 4-methoxycinnamic ester.

7. A preparation as claimed in claim 1, where the carrier C) is chosen from water, water-miscible liquids, hydrophobic components and mixtures thereof.

8. A method for protecting the human skin or the human hair against thermal radiation in the infrared wavelength range from 750 to 2500 nm and against UV radiation in the range from 280 to 449 nm, wherein a cosmetic or dermatological preparation as claimed in claim 1 is applied to the skin or the hair.

9. A method for reducing a warming of the skin or hair, wherein the skin or hair is treated with a cosmetic or dermatological preparation, comprising at least one cholesteric component which reflects in the infrared wavelength range from 750 nm to 2500 nm and at least one compound which protects against radiation in the ultraviolet wavelength range from 280 to 449 nm.

10. The method as claimed in claim 9, where the reduction in the warming is at least 20%.
